SEQUENCE LISTING (302 sequences)

SEQ ID NO: 1 - DNA, 13 bp, Artificial Sequence
  Description of Artificial Sequence Synthetic oligonucleotide sequence motif or oligomeric compound
gcagataagaaac

SEQ ID NO: 2 - DNA, 14 bp, Artificial Sequence
  Description of Artificial Sequence Synthetic oligonucleotide sequence motif or oligomeric compound
ggcagataagaaac

SEQ ID NO: 3 - DNA, 13 bp, Artificial Sequence
  Description of Artificial Sequence Synthetic oligonucleotide sequence motif or oligomeric compound
ttccgcaaaccca

SEQ ID NO: 4 - DNA, 14 bp, Artificial Sequence
  Description of Artificial Sequence Synthetic oligonucleotide sequence motif or oligomeric compound
attccgcaaaccca

SEQ ID NO: 5 - DNA, 15 bp, Artificial Sequence
  Description of Artificial Sequence Synthetic oligonucleotide sequence motif or oligomeric compound
cattccgcaaaccca

SEQ ID NO: 6 - DNA, 16 bp, Artificial Sequence
  Description of Artificial Sequence Synthetic oligonucleotide sequence motif or oligomeric compound
acattccgcaaaccca

SEQ ID NO: 7 - DNA, 12 bp, Artificial Sequence
  Description of Artificial Sequence Synthetic oligonucleotide sequence motif or oligomeric compound
ttccgcaaaccc

SEQ ID NO: 8 - DNA, 13 bp, Artificial Sequence
  Description of Artificial Sequence Synthetic oligonucleotide sequence motif or oligomeric compound
attccgcaaaccc

SEQ ID NO: 9 - DNA, 14 bp, Artificial Sequence
  Description of Artificial Sequence Synthetic oligonucleotide sequence motif or oligomeric compound
cattccgcaaaccc

SEQ ID NO: 10 - DNA, 15 bp, Artificial Sequence
  Description of Artificial Sequence Synthetic oligonucleotide sequence motif or oligomeric compound
acattccgcaaaccc

SEQ ID NO: 11 - DNA, 16 bp, Artificial Sequence
  Description of Artificial Sequence Synthetic oligonucleotide sequence motif or oligomeric compound
aacattccgcaaaccc

SEQ ID NO: 12 - DNA, 14 bp, Artificial Sequence
  Description of Artificial Sequence Synthetic oligonucleotide sequence motif or oligomeric compound
acattccgcaaacc

SEQ ID NO: 13 - DNA, 15 bp, Artificial Sequence
  Description of Artificial Sequence Synthetic oligonucleotide sequence motif or oligomeric compound
aacattccgcaaacc

SEQ ID NO: 14 - DNA, 16 bp, Artificial Sequence
  Description of Artificial Sequence Synthetic oligonucleotide sequence motif or oligomeric compound
taacattccgcaaacc

SEQ ID NO: 15 - DNA, 13 bp, Artificial Sequence
  Description of Artificial Sequence Synthetic oligonucleotide sequence motif or oligomeric compound
acattccgcaaac

SEQ ID NO: 16 - DNA, 14 bp, Artificial Sequence
  Description of Artificial Sequence Synthetic oligonucleotide sequence motif or oligomeric compound
aacattccgcaaac

SEQ ID NO: 17 - DNA, 15 bp, Artificial Sequence
  Description of Artificial Sequence Synthetic oligonucleotide sequence motif or oligomeric compound
taacattccgcaaac

SEQ ID NO: 18 - DNA, 16 bp, Artificial Sequence
  Description of Artificial Sequence Synthetic oligonucleotide sequence motif or oligomeric compound
ataacattccgcaaac

SEQ ID NO: 19 - DNA, 13 bp, Artificial Sequence
  Description of Artificial Sequence Synthetic oligonucleotide sequence motif or oligomeric compound
aacattccgcaaa

SEQ ID NO: 20 - DNA, 14 bp, Artificial Sequence
  Description of Artificial Sequence Synthetic oligonucleotide sequence motif or oligomeric compound
taacattccgcaaa

SEQ ID NO: 21 - DNA, 15 bp, Artificial Sequence
  Description of Artificial Sequence Synthetic oligonucleotide sequence motif or oligomeric compound
ataacattccgcaaa

SEQ ID NO: 22 - DNA, 16 bp, Artificial Sequence
  Description of Artificial Sequence Synthetic oligonucleotide sequence motif or oligomeric compound
aataacattccgcaaa

SEQ ID NO: 23 - DNA, 12 bp, Artificial Sequence
  Description of Artificial Sequence Synthetic oligonucleotide sequence motif or oligomeric compound
aacattccgcaa

SEQ ID NO: 24 - DNA, 13 bp, Artificial Sequence
  Description of Artificial Sequence Synthetic oligonucleotide sequence motif or oligomeric compound
taacattccgcaa

SEQ ID NO: 25 - DNA, 14 bp, Artificial Sequence
  Description of Artificial Sequence Synthetic oligonucleotide sequence motif or oligomeric compound
ataacattccgcaa

SEQ ID NO: 26 - DNA, 15 bp, Artificial Sequence
  Description of Artificial Sequence Synthetic oligonucleotide sequence motif or oligomeric compound
aataacattccgcaa

SEQ ID NO: 27 - DNA, 16 bp, Artificial Sequence
  Description of Artificial Sequence Synthetic oligonucleotide sequence motif or oligomeric compound
aaataacattccgcaa

SEQ ID NO: 28 - DNA, 12 bp, Artificial Sequence
  Description of Artificial Sequence Synthetic oligonucleotide sequence motif or oligomeric compound
taacattccgca

SEQ ID NO: 29 - DNA, 13 bp, Artificial Sequence
  Description of Artificial Sequence Synthetic oligonucleotide sequence motif or oligomeric compound
ataacattccgca

SEQ ID NO: 30 - DNA, 14 bp, Artificial Sequence
  Description of Artificial Sequence Synthetic oligonucleotide sequence motif or oligomeric compound
aataacattccgca

SEQ ID NO: 31 - DNA, 15 bp, Artificial Sequence
  Description of Artificial Sequence Synthetic oligonucleotide sequence motif or oligomeric compound
aaataacattccgca

SEQ ID NO: 32 - DNA, 16 bp, Artificial Sequence
  Description of Artificial Sequence Synthetic oligonucleotide sequence motif or oligomeric compound
taaataacattccgca

SEQ ID NO: 33 - DNA, 12 bp, Artificial Sequence
  Description of Artificial Sequence Synthetic oligonucleotide sequence motif or oligomeric compound
ataacattccgc

SEQ ID NO: 34 - DNA, 13 bp, Artificial Sequence
  Description of Artificial Sequence Synthetic oligonucleotide sequence motif or oligomeric compound
aataacattccgc

SEQ ID NO: 35 - DNA, 14 bp, Artificial Sequence
  Description of Artificial Sequence Synthetic oligonucleotide sequence motif or oligomeric compound
aaataacattccgc

SEQ ID NO: 36 - DNA, 15 bp, Artificial Sequence
  Description of Artificial Sequence Synthetic oligonucleotide sequence motif or oligomeric compound
taaataacattccgc

SEQ ID NO: 37 - DNA, 16 bp, Artificial Sequence
  Description of Artificial Sequence Synthetic oligonucleotide sequence motif or oligomeric compound
ataaataacattccgc

SEQ ID NO: 38 - DNA, 12 bp, Artificial Sequence
  Description of Artificial Sequence Synthetic oligonucleotide sequence motif or oligomeric compound
aataacattccg

SEQ ID NO: 39 - DNA, 13 bp, Artificial Sequence
  Description of Artificial Sequence Synthetic oligonucleotide sequence motif or oligomeric compound
aaataacattccg

SEQ ID NO: 40 - DNA, 14 bp, Artificial Sequence
  Description of Artificial Sequence Synthetic oligonucleotide sequence motif or oligomeric compound
taaataacattccg

SEQ ID NO: 41 - DNA, 15 bp, Artificial Sequence
  Description of Artificial Sequence Synthetic oligonucleotide sequence motif or oligomeric compound
ataaataacattccg

SEQ ID NO: 42 - DNA, 16 bp, Artificial Sequence
  Description of Artificial Sequence Synthetic oligonucleotide sequence motif or oligomeric compound
tataaataacattccg

SEQ ID NO: 43 - DNA, 15 bp, Artificial Sequence
  Description of Artificial Sequence Synthetic oligonucleotide sequence motif or oligomeric compound
tataaataacattcc

SEQ ID NO: 44 - DNA, 16 bp, Artificial Sequence
  Description of Artificial Sequence Synthetic oligonucleotide sequence motif or oligomeric compound
atataaataacattcc

SEQ ID NO: 45 - DNA, 14 bp, Artificial Sequence
  Description of Artificial Sequence Synthetic oligonucleotide sequence motif or oligomeric compound
tataaataacattc

SEQ ID NO: 46 - DNA, 15 bp, Artificial Sequence
  Description of Artificial Sequence Synthetic oligonucleotide sequence motif or oligomeric compound
atataaataacattc

SEQ ID NO: 47 - DNA, 16 bp, Artificial Sequence
  Description of Artificial Sequence Synthetic oligonucleotide sequence motif or oligomeric compound
gatataaataacattc

SEQ ID NO: 48 - DNA, 15 bp, Artificial Sequence
  Description of Artificial Sequence Synthetic oligonucleotide sequence motif or oligomeric compound
gatataaataacatt

SEQ ID NO: 49 - DNA, 16 bp, Artificial Sequence
  Description of Artificial Sequence Synthetic oligonucleotide sequence motif or oligomeric compound
tgatataaataacatt

SEQ ID NO: 50 - DNA, 15 bp, Artificial Sequence
  Description of Artificial Sequence Synthetic oligonucleotide sequence motif or oligomeric compound
tgatataaataacat

SEQ ID NO: 51 - DNA, 16 bp, Artificial Sequence
  Description of Artificial Sequence Synthetic oligonucleotide sequence motif or oligomeric compound
ttgatataaataacat

SEQ ID NO: 52 - DNA, 14 bp, Artificial Sequence
  Description of Artificial Sequence Synthetic oligonucleotide sequence motif or oligomeric compound
tgatataaataaca

SEQ ID NO: 53 - DNA, 15 bp, Artificial Sequence
  Description of Artificial Sequence Synthetic oligonucleotide sequence motif or oligomeric compound
ttgatataaataaca

SEQ ID NO: 54 - DNA, 16 bp, Artificial Sequence
  Description of Artificial Sequence Synthetic oligonucleotide sequence motif or oligomeric compound
attgatataaataaca

SEQ ID NO: 55 - DNA, 13 bp, Artificial Sequence
  Description of Artificial Sequence Synthetic oligonucleotide sequence motif or oligomeric compound
tgatataaataac

SEQ ID NO: 56 - DNA, 14 bp, Artificial Sequence
  Description of Artificial Sequence Synthetic oligonucleotide sequence motif or oligomeric compound
ttgatataaataac

SEQ ID NO: 57 - DNA, 15 bp, Artificial Sequence
  Description of Artificial Sequence Synthetic oligonucleotide sequence motif or oligomeric compound
attgatataaataac

SEQ ID NO: 58 - DNA, 16 bp, Artificial Sequence
  Description of Artificial Sequence Synthetic oligonucleotide sequence motif or oligomeric compound
cattgatataaataac

SEQ ID NO: 59 - DNA, 14 bp, Artificial Sequence
  Description of Artificial Sequence Synthetic oligonucleotide sequence motif or oligomeric compound
attgatataaataa

SEQ ID NO: 60 - DNA, 15 bp, Artificial Sequence
  Description of Artificial Sequence Synthetic oligonucleotide sequence motif or oligomeric compound
cattgatataaataa

SEQ ID NO: 61 - DNA, 16 bp, Artificial Sequence
  Description of Artificial Sequence Synthetic oligonucleotide sequence motif or oligomeric compound
tcattgatataaataa

SEQ ID NO: 62 - DNA, 16 bp, Artificial Sequence
  Description of Artificial Sequence Synthetic oligonucleotide sequence motif or oligomeric compound
gtttcattgatataaa

SEQ ID NO: 63 - DNA, 15 bp, Artificial Sequence
  Description of Artificial Sequence Synthetic oligonucleotide sequence motif or oligomeric compound
gtttcattgatataa

SEQ ID NO: 64 - DNA, 14 bp, Artificial Sequence
  Description of Artificial Sequence Synthetic oligonucleotide sequence motif or oligomeric compound
gtttcattgatata

SEQ ID NO: 65 - DNA, 13 bp, Artificial Sequence
  Description of Artificial Sequence Synthetic oligonucleotide sequence motif or oligomeric compound
acatgcccttatg

SEQ ID NO: 66 - DNA, 14 bp, Artificial Sequence
  Description of Artificial Sequence Synthetic oligonucleotide sequence motif or oligomeric compound
aacatgcccttatg

SEQ ID NO: 67 - DNA, 15 bp, Artificial Sequence
  Description of Artificial Sequence Synthetic oligonucleotide sequence motif or oligomeric compound
aaacatgcccttatg

SEQ ID NO: 68 - DNA, 16 bp, Artificial Sequence
  Description of Artificial Sequence Synthetic oligonucleotide sequence motif or oligomeric compound
caaacatgcccttatg

SEQ ID NO: 69 - DNA, 13 bp, Artificial Sequence
  Description of Artificial Sequence Synthetic oligonucleotide sequence motif or oligomeric compound
aacatgcccttat

SEQ ID NO: 70 - DNA, 14 bp, Artificial Sequence
  Description of Artificial Sequence Synthetic oligonucleotide sequence motif or oligomeric compound
aaacatgcccttat

SEQ ID NO: 71 - DNA, 15 bp, Artificial Sequence
  Description of Artificial Sequence Synthetic oligonucleotide sequence motif or oligomeric compound
caaacatgcccttat

SEQ ID NO: 72 - DNA, 14 bp, Artificial Sequence
  Description of Artificial Sequence Synthetic oligonucleotide sequence motif or oligomeric compound
caaacatgccctta

SEQ ID NO: 73 - DNA, 13 bp, Artificial Sequence
  Description of Artificial Sequence Synthetic oligonucleotide sequence motif or oligomeric compound
caaacatgccctt

SEQ ID NO: 74 - DNA, 13 bp, Artificial Sequence
  Description of Artificial Sequence Synthetic oligonucleotide sequence motif or oligomeric compound
caattgcctcttg

SEQ ID NO: 75 - DNA, 13 bp, Artificial Sequence
  Description of Artificial Sequence Synthetic oligonucleotide sequence motif or oligomeric compound
agaagctgttgaa

SEQ ID NO: 76 - DNA, 14 bp, Artificial Sequence
  Description of Artificial Sequence Synthetic oligonucleotide sequence motif or oligomeric compound
aagaagctgttgaa

SEQ ID NO: 77 - DNA, 14 bp, Artificial Sequence
  Description of Artificial Sequence Synthetic oligonucleotide sequence motif or oligomeric compound
cgtctcagttgcag

SEQ ID NO: 78 - DNA, 15 bp, Artificial Sequence
  Description of Artificial Sequence Synthetic oligonucleotide sequence motif or oligomeric compound
tcgtctcagttgcag

SEQ ID NO: 79 - DNA, 16 bp, Artificial Sequence
  Description of Artificial Sequence Synthetic oligonucleotide sequence motif or oligomeric compound
ttcgtctcagttgcag

SEQ ID NO: 80 - DNA, 13 bp, Artificial Sequence
  Description of Artificial Sequence Synthetic oligonucleotide sequence motif or oligomeric compound
cgtctcagttgca

SEQ ID NO: 81 - DNA, 14 bp, Artificial Sequence
  Description of Artificial Sequence Synthetic oligonucleotide sequence motif or oligomeric compound
tcgtctcagttgca

SEQ ID NO: 82 - DNA, 15 bp, Artificial Sequence
  Description of Artificial Sequence Synthetic oligonucleotide sequence motif or oligomeric compound
ttcgtctcagttgca

SEQ ID NO: 83 - DNA, 13 bp, Artificial Sequence
  Description of Artificial Sequence Synthetic oligonucleotide sequence motif or oligomeric compound
tcgtctcagttgc

SEQ ID NO: 84 - DNA, 14 bp, Artificial Sequence
  Description of Artificial Sequence Synthetic oligonucleotide sequence motif or oligomeric compound
ttcgtctcagttgc

SEQ ID NO: 85 - DNA, 12 bp, Artificial Sequence
  Description of Artificial Sequence Synthetic oligonucleotide sequence motif or oligomeric compound
tcgtctcagttg

SEQ ID NO: 86 - DNA, 13 bp, Artificial Sequence
  Description of Artificial Sequence Synthetic oligonucleotide sequence motif or oligomeric compound
ttcgtctcagttg

SEQ ID NO: 87 - DNA, 12 bp, Artificial Sequence
  Description of Artificial Sequence Synthetic oligonucleotide sequence motif or oligomeric compound
ttcgtctcagtt

SEQ ID NO: 88 - DNA, 13 bp, Artificial Sequence
  Description of Artificial Sequence Synthetic oligonucleotide sequence motif or oligomeric compound
tgagattattgcc

SEQ ID NO: 89 - DNA, 14 bp, Artificial Sequence
  Description of Artificial Sequence Synthetic oligonucleotide sequence motif or oligomeric compound
ttgagattattgcc

SEQ ID NO: 90 - DNA, 15 bp, Artificial Sequence
  Description of Artificial Sequence Synthetic oligonucleotide sequence motif or oligomeric compound
gttgagattattgcc

SEQ ID NO: 91 - DNA, 16 bp, Artificial Sequence
  Description of Artificial Sequence Synthetic oligonucleotide sequence motif or oligomeric compound
tgttgagattattgcc

SEQ ID NO: 92 - DNA, 14 bp, Artificial Sequence
  Description of Artificial Sequence Synthetic oligonucleotide sequence motif or oligomeric compound
gttgagattattgc

SEQ ID NO: 93 - DNA, 15 bp, Artificial Sequence
  Description of Artificial Sequence Synthetic oligonucleotide sequence motif or oligomeric compound
tgttgagattattgc

SEQ ID NO: 94 - DNA, 16 bp, Artificial Sequence
  Description of Artificial Sequence Synthetic oligonucleotide sequence motif or oligomeric compound
atgttgagattattgc

SEQ ID NO: 95 - DNA, 13 bp, Artificial Sequence
  Description of Artificial Sequence Synthetic oligonucleotide sequence motif or oligomeric compound
gttgagattattg

SEQ ID NO: 96 - DNA, 14 bp, Artificial Sequence
  Description of Artificial Sequence Synthetic oligonucleotide sequence motif or oligomeric compound
tgttgagattattg

SEQ ID NO: 97 - DNA, 15 bp, Artificial Sequence
  Description of Artificial Sequence Synthetic oligonucleotide sequence motif or oligomeric compound
atgttgagattattg

SEQ ID NO: 98 - DNA, 16 bp, Artificial Sequence
  Description of Artificial Sequence Synthetic oligonucleotide sequence motif or oligomeric compound
gatgttgagattattg

SEQ ID NO: 99 - DNA, 14 bp, Artificial Sequence
  Description of Artificial Sequence Synthetic oligonucleotide sequence motif or oligomeric compound
atgttgagattatt

SEQ ID NO: 100 - DNA, 15 bp, Artificial Sequence
  Description of Artificial Sequence Synthetic oligonucleotide sequence motif or oligomeric compound
gatgttgagattatt

SEQ ID NO: 101 - DNA, 16 bp, Artificial Sequence
  Description of Artificial Sequence Synthetic oligonucleotide sequence motif or oligomeric compound
ggatgttgagattatt

SEQ ID NO: 102 - DNA, 13 bp, Artificial Sequence
  Description of Artificial Sequence Synthetic oligonucleotide sequence motif or oligomeric compound
atgttgagattat

SEQ ID NO: 103 - DNA, 14 bp, Artificial Sequence
  Description of Artificial Sequence Synthetic oligonucleotide sequence motif or oligomeric compound
gatgttgagattat

SEQ ID NO: 104 - DNA, 15 bp, Artificial Sequence
  Description of Artificial Sequence Synthetic oligonucleotide sequence motif or oligomeric compound
ggatgttgagattat

SEQ ID NO: 105 - DNA, 16 bp, Artificial Sequence
  Description of Artificial Sequence Synthetic oligonucleotide sequence motif or oligomeric compound
gggatgttgagattat

SEQ ID NO: 106 - DNA, 13 bp, Artificial Sequence
  Description of Artificial Sequence Synthetic oligonucleotide sequence motif or oligomeric compound
gatgttgagatta

SEQ ID NO: 107 - DNA, 14 bp, Artificial Sequence
  Description of Artificial Sequence Synthetic oligonucleotide sequence motif or oligomeric compound
ggatgttgagatta

SEQ ID NO: 108 - DNA, 15 bp, Artificial Sequence
  Description of Artificial Sequence Synthetic oligonucleotide sequence motif or oligomeric compound
gggatgttgagatta

SEQ ID NO: 109 - DNA, 14 bp, Artificial Sequence
  Description of Artificial Sequence Synthetic oligonucleotide sequence motif or oligomeric compound
gggatgttgagatt

SEQ ID NO: 110 - DNA, 12 bp, Artificial Sequence
  Description of Artificial Sequence Synthetic oligonucleotide sequence motif or oligomeric compound
ttcatcgagcct

SEQ ID NO: 111 - DNA, 13 bp, Artificial Sequence
  Description of Artificial Sequence Synthetic oligonucleotide sequence motif or oligomeric compound
tttcatcgagcct

SEQ ID NO: 112 - DNA, 14 bp, Artificial Sequence
  Description of Artificial Sequence Synthetic oligonucleotide sequence motif or oligomeric compound
gtttcatcgagcct

SEQ ID NO: 113 - DNA, 13 bp, Artificial Sequence
  Description of Artificial Sequence Synthetic oligonucleotide sequence motif or oligomeric compound
gtttcatcgagcc

SEQ ID NO: 114 - DNA, 12 bp, Artificial Sequence
  Description of Artificial Sequence Synthetic oligonucleotide sequence motif or oligomeric compound
gtttcatcgagc

SEQ ID NO: 115 - DNA, 13 bp, Artificial Sequence
  Description of Artificial Sequence Synthetic oligonucleotide sequence motif or oligomeric compound
agtgaccttcgat

SEQ ID NO: 116 - DNA, 14 bp, Artificial Sequence
  Description of Artificial Sequence Synthetic oligonucleotide sequence motif or oligomeric compound
tagtgaccttcgat

SEQ ID NO: 117 - DNA, 15 bp, Artificial Sequence
  Description of Artificial Sequence Synthetic oligonucleotide sequence motif or oligomeric compound
gtagtgaccttcgat

SEQ ID NO: 118 - DNA, 16 bp, Artificial Sequence
  Description of Artificial Sequence Synthetic oligonucleotide sequence motif or oligomeric compound
tgtagtgaccttcgat

SEQ ID NO: 119 - DNA, 13 bp, Artificial Sequence
  Description of Artificial Sequence Synthetic oligonucleotide sequence motif or oligomeric compound
tagtgaccttcga

SEQ ID NO: 120 - DNA, 14 bp, Artificial Sequence
  Description of Artificial Sequence Synthetic oligonucleotide sequence motif or oligomeric compound
gtagtgaccttcga

SEQ ID NO: 121 - DNA, 15 bp, Artificial Sequence
  Description of Artificial Sequence Synthetic oligonucleotide sequence motif or oligomeric compound
tgtagtgaccttcga

SEQ ID NO: 122 - DNA, 16 bp, Artificial Sequence
  Description of Artificial Sequence Synthetic oligonucleotide sequence motif or oligomeric compound
ttgtagtgaccttcga

SEQ ID NO: 123 - DNA, 12 bp, Artificial Sequence
  Description of Artificial Sequence Synthetic oligonucleotide sequence motif or oligomeric compound
tagtgaccttcg

SEQ ID NO: 124 - DNA, 13 bp, Artificial Sequence
  Description of Artificial Sequence Synthetic oligonucleotide sequence motif or oligomeric compound
gtagtgaccttcg

SEQ ID NO: 125 - DNA, 14 bp, Artificial Sequence
  Description of Artificial Sequence Synthetic oligonucleotide sequence motif or oligomeric compound
tgtagtgaccttcg

SEQ ID NO: 126 - DNA, 15 bp, Artificial Sequence
  Description of Artificial Sequence Synthetic oligonucleotide sequence motif or oligomeric compound
ttgtagtgaccttcg

SEQ ID NO: 127 - DNA, 16 bp, Artificial Sequence
  Description of Artificial Sequence Synthetic oligonucleotide sequence motif or oligomeric compound
attgtagtgaccttcg

SEQ ID NO: 128 - DNA, 14 bp, Artificial Sequence
  Description of Artificial Sequence Synthetic oligonucleotide sequence motif or oligomeric compound
ttgtagtgaccttc

SEQ ID NO: 129 - DNA, 15 bp, Artificial Sequence
  Description of Artificial Sequence Synthetic oligonucleotide sequence motif or oligomeric compound
attgtagtgaccttc

SEQ ID NO: 130 - DNA, 14 bp, Artificial Sequence
  Description of Artificial Sequence Synthetic oligonucleotide sequence motif or oligomeric compound
attgtagtgacctt

SEQ ID NO: 131 - DNA, 15 bp, Artificial Sequence
  Description of Artificial Sequence Synthetic oligonucleotide sequence motif or oligomeric compound
ttctaaatattcctt

SEQ ID NO: 132 - DNA, 14 bp, Artificial Sequence
  Description of Artificial Sequence Synthetic oligonucleotide sequence motif or oligomeric compound
ttctaaatattcct

SEQ ID NO: 133 - DNA, 13 bp, Artificial Sequence
  Description of Artificial Sequence Synthetic oligonucleotide sequence motif or oligomeric compound
ctgtagaggagca

SEQ ID NO: 134 - DNA, 14 bp, Artificial Sequence
  Description of Artificial Sequence Synthetic oligonucleotide sequence motif or oligomeric compound
gcctgtgtctgtag

SEQ ID NO: 135 - DNA, 15 bp, Artificial Sequence
  Description of Artificial Sequence Synthetic oligonucleotide sequence motif or oligomeric compound
tgcctgtgtctgtag

SEQ ID NO: 136 - DNA, 14 bp, Artificial Sequence
  Description of Artificial Sequence Synthetic oligonucleotide sequence motif or oligomeric compound
tgcctgtgtctgta

SEQ ID NO: 137 - DNA, 15 bp, Artificial Sequence
  Description of Artificial Sequence Synthetic oligonucleotide sequence motif or oligomeric compound
ctgcctgtgtctgta

SEQ ID NO: 138 - DNA, 16 bp, Artificial Sequence
  Description of Artificial Sequence Synthetic oligonucleotide sequence motif or oligomeric compound
cctgcctgtgtctgta

SEQ ID NO: 139 - DNA, 16 bp, Artificial Sequence
  Description of Artificial Sequence Synthetic oligonucleotide sequence motif or oligomeric compound
ccctgcctgtgtctgt

SEQ ID NO: 140 - DNA, 15 bp, Artificial Sequence
  Description of Artificial Sequence Synthetic oligonucleotide sequence motif or oligomeric compound
ccctgcctgtgtctg

SEQ ID NO: 141 - DNA, 16 bp, Artificial Sequence
  Description of Artificial Sequence Synthetic oligonucleotide sequence motif or oligomeric compound
tccctgcctgtgtctg

SEQ ID NO: 142 - DNA, 16 bp, Artificial Sequence
  Description of Artificial Sequence Synthetic oligonucleotide sequence motif or oligomeric compound
ttccctgcctgtgtct

SEQ ID NO: 143 - DNA, 15 bp, Artificial Sequence
  Description of Artificial Sequence Synthetic oligonucleotide sequence motif or oligomeric compound
ttccctgcctgtgtc

SEQ ID NO: 144 - DNA, 16 bp, Artificial Sequence
  Description of Artificial Sequence Synthetic oligonucleotide sequence motif or oligomeric compound
attccctgcctgtgtc

SEQ ID NO: 145 - DNA, 14 bp, Artificial Sequence
  Description of Artificial Sequence Synthetic oligonucleotide sequence motif or oligomeric compound
ttccctgcctgtgt

SEQ ID NO: 146 - DNA, 15 bp, Artificial Sequence
  Description of Artificial Sequence Synthetic oligonucleotide sequence motif or oligomeric compound
attccctgcctgtgt

SEQ ID NO: 147 - DNA, 14 bp, Artificial Sequence
  Description of Artificial Sequence Synthetic oligonucleotide sequence motif or oligomeric compound
attccctgcctgtg

SEQ ID NO: 148 - DNA, 14 bp, Artificial Sequence
  Description of Artificial Sequence Synthetic oligonucleotide sequence motif or oligomeric compound
tcacaaagaagtct

SEQ ID NO: 149 - DNA, 15 bp, Artificial Sequence
  Description of Artificial Sequence Synthetic oligonucleotide sequence motif or oligomeric compound
atcacaaagaagtct

SEQ ID NO: 150 - DNA, 16 bp, Artificial Sequence
  Description of Artificial Sequence Synthetic oligonucleotide sequence motif or oligomeric compound
catcacaaagaagtct

SEQ ID NO: 151 - DNA, 14 bp, Artificial Sequence
  Description of Artificial Sequence Synthetic oligonucleotide sequence motif or oligomeric compound
atcacaaagaagtc

SEQ ID NO: 152 - DNA, 15 bp, Artificial Sequence
  Description of Artificial Sequence Synthetic oligonucleotide sequence motif or oligomeric compound
catcacaaagaagtc

SEQ ID NO: 153 - DNA, 16 bp, Artificial Sequence
  Description of Artificial Sequence Synthetic oligonucleotide sequence motif or oligomeric compound
tcatcacaaagaagtc

SEQ ID NO: 154 - DNA, 13 bp, Artificial Sequence
  Description of Artificial Sequence Synthetic oligonucleotide sequence motif or oligomeric compound
atcacaaagaagt

SEQ ID NO: 155 - DNA, 14 bp, Artificial Sequence
  Description of Artificial Sequence Synthetic oligonucleotide sequence motif or oligomeric compound
catcacaaagaagt

SEQ ID NO: 156 - DNA, 15 bp, Artificial Sequence
  Description of Artificial Sequence Synthetic oligonucleotide sequence motif or oligomeric compound
tcatcacaaagaagt

SEQ ID NO: 157 - DNA, 16 bp, Artificial Sequence
  Description of Artificial Sequence Synthetic oligonucleotide sequence motif or oligomeric compound
ttcatcacaaagaagt

SEQ ID NO: 158 - DNA, 15 bp, Artificial Sequence
  Description of Artificial Sequence Synthetic oligonucleotide sequence motif or oligomeric compound
ttcatcacaaagaag

SEQ ID NO: 159 - DNA, 14 bp, Artificial Sequence
  Description of Artificial Sequence Synthetic oligonucleotide sequence motif or oligomeric compound
acatcttctgaatt

SEQ ID NO: 160 - DNA, 14 bp, Artificial Sequence
  Description of Artificial Sequence Synthetic oligonucleotide sequence motif or oligomeric compound
ggtgattgtgacac

SEQ ID NO: 161 - DNA, 15 bp, Artificial Sequence
  Description of Artificial Sequence Synthetic oligonucleotide sequence motif or oligomeric compound
gggtgattgtgacac

SEQ ID NO: 162 - DNA, 16 bp, Artificial Sequence
  Description of Artificial Sequence Synthetic oligonucleotide sequence motif or oligomeric compound
tgggtgattgtgacac

SEQ ID NO: 163 - DNA, 13 bp, Artificial Sequence
  Description of Artificial Sequence Synthetic oligonucleotide sequence motif or oligomeric compound
ggtgattgtgaca

SEQ ID NO: 164 - DNA, 14 bp, Artificial Sequence
  Description of Artificial Sequence Synthetic oligonucleotide sequence motif or oligomeric compound
gggtgattgtgaca

SEQ ID NO: 165 - DNA, 15 bp, Artificial Sequence
  Description of Artificial Sequence Synthetic oligonucleotide sequence motif or oligomeric compound
tgggtgattgtgaca

SEQ ID NO: 166 - DNA, 16 bp, Artificial Sequence
  Description of Artificial Sequence Synthetic oligonucleotide sequence motif or oligomeric compound
gtgggtgattgtgaca

SEQ ID NO: 167 - DNA, 13 bp, Artificial Sequence
  Description of Artificial Sequence Synthetic oligonucleotide sequence motif or oligomeric compound
gggtgattgtgac

SEQ ID NO: 168 - DNA, 14 bp, Artificial Sequence
  Description of Artificial Sequence Synthetic oligonucleotide sequence motif or oligomeric compound
tgggtgattgtgac

SEQ ID NO: 169 - DNA, 15 bp, Artificial Sequence
  Description of Artificial Sequence Synthetic oligonucleotide sequence motif or oligomeric compound
gtgggtgattgtgac

SEQ ID NO: 170 - DNA, 16 bp, Artificial Sequence
  Description of Artificial Sequence Synthetic oligonucleotide sequence motif or oligomeric compound
tgtgggtgattgtgac

SEQ ID NO: 171 - DNA, 12 bp, Artificial Sequence
  Description of Artificial Sequence Synthetic oligonucleotide sequence motif or oligomeric compound
ggtgattgtgac

SEQ ID NO: 172 - DNA, 13 bp, Artificial Sequence
  Description of Artificial Sequence Synthetic oligonucleotide sequence motif or oligomeric compound
tgggtgattgtga

SEQ ID NO: 173 - DNA, 14 bp, Artificial Sequence
  Description of Artificial Sequence Synthetic oligonucleotide sequence motif or oligomeric compound
gtgggtgattgtga

SEQ ID NO: 174 - DNA, 15 bp, Artificial Sequence
  Description of Artificial Sequence Synthetic oligonucleotide sequence motif or oligomeric compound
tgtgggtgattgtga

SEQ ID NO: 175 - DNA, 16 bp, Artificial Sequence
  Description of Artificial Sequence Synthetic oligonucleotide sequence motif or oligomeric compound
gtgtgggtgattgtga

SEQ ID NO: 176 - DNA, 15 bp, Artificial Sequence
  Description of Artificial Sequence Synthetic oligonucleotide sequence motif or oligomeric compound
gtgtgggtgattgtg

SEQ ID NO: 177 - DNA, 13 bp, Artificial Sequence
  Description of Artificial Sequence Synthetic oligonucleotide sequence motif or oligomeric compound
gggacagttgtgc

SEQ ID NO: 178 - DNA, 14 bp, Artificial Sequence
  Description of Artificial Sequence Synthetic oligonucleotide sequence motif or oligomeric compound
tagaagttgagttc

SEQ ID NO: 179 - DNA, 15 bp, Artificial Sequence
  Description of Artificial Sequence Synthetic oligonucleotide sequence motif or oligomeric compound
gtagaagttgagttc

SEQ ID NO: 180 - DNA, 16 bp, Artificial Sequence
  Description of Artificial Sequence Synthetic oligonucleotide sequence motif or oligomeric compound
tgtagaagttgagttc

SEQ ID NO: 181 - DNA, 14 bp, Artificial Sequence
  Description of Artificial Sequence Synthetic oligonucleotide sequence motif or oligomeric compound
gtagaagttgagtt

SEQ ID NO: 182 - DNA, 15 bp, Artificial Sequence
  Description of Artificial Sequence Synthetic oligonucleotide sequence motif or oligomeric compound
tgtagaagttgagtt

SEQ ID NO: 183 - DNA, 16 bp, Artificial Sequence
  Description of Artificial Sequence Synthetic oligonucleotide sequence motif or oligomeric compound
ctgtagaagttgagtt

SEQ ID NO: 184 - DNA, 15 bp, Artificial Sequence
  Description of Artificial Sequence Synthetic oligonucleotide sequence motif or oligomeric compound
ctgtagaagttgagt

SEQ ID NO: 185 - DNA, 16 bp, Artificial Sequence
  Description of Artificial Sequence Synthetic oligonucleotide sequence motif or oligomeric compound
gctgtagaagttgagt

SEQ ID NO: 186 - DNA, 14 bp, Artificial Sequence
  Description of Artificial Sequence Synthetic oligonucleotide sequence motif or oligomeric compound
ctgtagaagttgag

SEQ ID NO: 187 - DNA, 15 bp, Artificial Sequence
  Description of Artificial Sequence Synthetic oligonucleotide sequence motif or oligomeric compound
gctgtagaagttgag

SEQ ID NO: 188 - DNA, 14 bp, Artificial Sequence
  Description of Artificial Sequence Synthetic oligonucleotide sequence motif or oligomeric compound
gctgtagaagttga

SEQ ID NO: 189 - DNA, 13 bp, Artificial Sequence
  Description of Artificial Sequence Synthetic oligonucleotide sequence motif or oligomeric compound
caagctatgatgg

SEQ ID NO: 190 - DNA, 14 bp, Artificial Sequence
  Description of Artificial Sequence Synthetic oligonucleotide sequence motif or oligomeric compound
gcaagctatgatgg

SEQ ID NO: 191 - DNA, 15 bp, Artificial Sequence
  Description of Artificial Sequence Synthetic oligonucleotide sequence motif or oligomeric compound
tgcaagctatgatgg

SEQ ID NO: 192 - DNA, 16 bp, Artificial Sequence
  Description of Artificial Sequence Synthetic oligonucleotide sequence motif or oligomeric compound
ctgcaagctatgatgg

SEQ ID NO: 193 - DNA, 14 bp, Artificial Sequence
  Description of Artificial Sequence Synthetic oligonucleotide sequence motif or oligomeric compound
tgcaagctatgatg

SEQ ID NO: 194 - DNA, 15 bp, Artificial Sequence
  Description of Artificial Sequence Synthetic oligonucleotide sequence motif or oligomeric compound
ctgcaagctatgatg

SEQ ID NO: 195 - DNA, 13 bp, Artificial Sequence
  Description of Artificial Sequence Synthetic oligonucleotide sequence motif or oligomeric compound
tgcaagctatgat

SEQ ID NO: 196 - DNA, 14 bp, Artificial Sequence
  Description of Artificial Sequence Synthetic oligonucleotide sequence motif or oligomeric compound
ctgcaagctatgat

SEQ ID NO: 197 - DNA, 14 bp, Artificial Sequence
  Description of Artificial Sequence Synthetic oligonucleotide sequence motif or oligomeric compound
ctcctggctgatca

SEQ ID NO: 198 - DNA, 14 bp, Artificial Sequence
  Description of Artificial Sequence Synthetic oligonucleotide sequence motif or oligomeric compound
aaggtagcacaggc

SEQ ID NO: 199 - DNA, 15 bp, Artificial Sequence
  Description of Artificial Sequence Synthetic oligonucleotide sequence motif or oligomeric compound
gaaggtagcacaggc

SEQ ID NO: 200 - DNA, 16 bp, Artificial Sequence
  Description of Artificial Sequence Synthetic oligonucleotide sequence motif or oligomeric compound
agaaggtagcacaggc

SEQ ID NO: 201 - DNA, 14 bp, Artificial Sequence
  Description of Artificial Sequence Synthetic oligonucleotide sequence motif or oligomeric compound
gaaggtagcacagg

SEQ ID NO: 202 - DNA, 15 bp, Artificial Sequence
  Description of Artificial Sequence Synthetic oligonucleotide sequence motif or oligomeric compound
agaaggtagcacagg

SEQ ID NO: 203 - DNA, 16 bp, Artificial Sequence
  Description of Artificial Sequence Synthetic oligonucleotide sequence motif or oligomeric compound
gagaaggtagcacagg

SEQ ID NO: 204 - DNA, 14 bp, Artificial Sequence
  Description of Artificial Sequence Synthetic oligonucleotide sequence motif or oligomeric compound
agaaggtagcacag

SEQ ID NO: 205 - DNA, 15 bp, Artificial Sequence
  Description of Artificial Sequence Synthetic oligonucleotide sequence motif or oligomeric compound
gagaaggtagcacag

SEQ ID NO: 206 - DNA, 16 bp, Artificial Sequence
  Description of Artificial Sequence Synthetic oligonucleotide sequence motif or oligomeric compound
agagaaggtagcacag

SEQ ID NO: 207 - DNA, 13 bp, Artificial Sequence
  Description of Artificial Sequence Synthetic oligonucleotide sequence motif or oligomeric compound
agaaggtagcaca

SEQ ID NO: 208 - DNA, 14 bp, Artificial Sequence
  Description of Artificial Sequence Synthetic oligonucleotide sequence motif or oligomeric compound
gagaaggtagcaca

SEQ ID NO: 209 - DNA, 15 bp, Artificial Sequence
  Description of Artificial Sequence Synthetic oligonucleotide sequence motif or oligomeric compound
agagaaggtagcaca

SEQ ID NO: 210 - DNA, 16 bp, Artificial Sequence
  Description of Artificial Sequence Synthetic oligonucleotide sequence motif or oligomeric compound
gagagaaggtagcaca

SEQ ID NO: 211 - DNA, 12 bp, Artificial Sequence
  Description of Artificial Sequence Synthetic oligonucleotide sequence motif or oligomeric compound
agaaggtagcac

SEQ ID NO: 212 - DNA, 13 bp, Artificial Sequence
  Description of Artificial Sequence Synthetic oligonucleotide sequence motif or oligomeric compound
gagaaggtagcac

SEQ ID NO: 213 - DNA, 14 bp, Artificial Sequence
  Description of Artificial Sequence Synthetic oligonucleotide sequence motif or oligomeric compound
agagaaggtagcac

SEQ ID NO: 214 - DNA, 15 bp, Artificial Sequence
  Description of Artificial Sequence Synthetic oligonucleotide sequence motif or oligomeric compound
gagagaaggtagcac

SEQ ID NO: 215 - DNA, 14 bp, Artificial Sequence
  Description of Artificial Sequence Synthetic oligonucleotide sequence motif or oligomeric compound
gagagaaggtagca

SEQ ID NO: 216 - DNA, 13 bp, Artificial Sequence
  Description of Artificial Sequence Synthetic oligonucleotide sequence motif or oligomeric compound
gagagaaggtagc

SEQ ID NO: 217 - DNA, 14 bp, Artificial Sequence
  Description of Artificial Sequence Synthetic oligonucleotide sequence motif or oligomeric compound
gaatgccatactgg

SEQ ID NO: 218 - DNA, 15 bp, Artificial Sequence
  Description of Artificial Sequence Synthetic oligonucleotide sequence motif or oligomeric compound
agaatgccatactgg

SEQ ID NO: 219 - DNA, 14 bp, Artificial Sequence
  Description of Artificial Sequence Synthetic oligonucleotide sequence motif or oligomeric compound
agaatgccatactg

SEQ ID NO: 220 - DNA, 15 bp, Artificial Sequence
  Description of Artificial Sequence Synthetic oligonucleotide sequence motif or oligomeric compound
atcttcctggtcatc

SEQ ID NO: 221 - DNA, 14 bp, Artificial Sequence
  Description of Artificial Sequence Synthetic oligonucleotide sequence motif or oligomeric compound
ttgtcccactgctg

SEQ ID NO: 222 - DNA, 15 bp, Artificial Sequence
  Description of Artificial Sequence Synthetic oligonucleotide sequence motif or oligomeric compound
cttgtcccactgctg

SEQ ID NO: 223 - DNA, 16 bp, Artificial Sequence
  Description of Artificial Sequence Synthetic oligonucleotide sequence motif or oligomeric compound
tcttgtcccactgctg

SEQ ID NO: 224 - DNA, 14 bp, Artificial Sequence
  Description of Artificial Sequence Synthetic oligonucleotide sequence motif or oligomeric compound
cttgtcccactgct

SEQ ID NO: 225 - DNA, 15 bp, Artificial Sequence
  Description of Artificial Sequence Synthetic oligonucleotide sequence motif or oligomeric compound
tcttgtcccactgct

SEQ ID NO: 226 - DNA, 16 bp, Artificial Sequence
  Description of Artificial Sequence Synthetic oligonucleotide sequence motif or oligomeric compound
ttcttgtcccactgct

SEQ ID NO: 227 - DNA, 16 bp, Artificial Sequence
  Description of Artificial Sequence Synthetic oligonucleotide sequence motif or oligomeric compound
gcttcttgtcccactg

SEQ ID NO: 228 - DNA, 15 bp, Artificial Sequence
  Description of Artificial Sequence Synthetic oligonucleotide sequence motif or oligomeric compound
gcttcttgtcccact

SEQ ID NO: 229 - DNA, 16 bp, Artificial Sequence
  Description of Artificial Sequence Synthetic oligonucleotide sequence motif or oligomeric compound
agcttcttgtcccact

SEQ ID NO: 230 - DNA, 14 bp, Artificial Sequence
  Description of Artificial Sequence Synthetic oligonucleotide sequence motif or oligomeric compound
gcttcttgtcccac

SEQ ID NO: 231 - DNA, 15 bp, Artificial Sequence
  Description of Artificial Sequence Synthetic oligonucleotide sequence motif or oligomeric compound
agcttcttgtcccac

SEQ ID NO: 232 - DNA, 16 bp, Artificial Sequence
  Description of Artificial Sequence Synthetic oligonucleotide sequence motif or oligomeric compound
aagcttcttgtcccac

SEQ ID NO: 233 - DNA, 14 bp, Artificial Sequence
  Description of Artificial Sequence Synthetic oligonucleotide sequence motif or oligomeric compound
agcttcttgtccca

SEQ ID NO: 234 - DNA, 15 bp, Artificial Sequence
  Description of Artificial Sequence Synthetic oligonucleotide sequence motif or oligomeric compound
aagcttcttgtccca

SEQ ID NO: 235 - DNA, 14 bp, Artificial Sequence
  Description of Artificial Sequence Synthetic oligonucleotide sequence motif or oligomeric compound
aagcttcttgtccc

SEQ ID NO: 236 - DNA, 13 bp, Artificial Sequence
  Description of Artificial Sequence Synthetic oligonucleotide sequence motif or oligomeric compound
aagcttcttgtcc

SEQ ID NO: 237 - DNA, 15 bp, Artificial Sequence
  Description of Artificial Sequence Synthetic oligonucleotide sequence motif or oligomeric compound
actgtcttcatcttc

SEQ ID NO: 238 - DNA, 16 bp, Artificial Sequence
  Description of Artificial Sequence Synthetic oligonucleotide sequence motif or oligomeric compound
cactgtcttcatcttc

SEQ ID NO: 239 - DNA, 16 bp, Artificial Sequence
  Description of Artificial Sequence Synthetic oligonucleotide sequence motif or oligomeric compound
agtcactgtcttcatc

SEQ ID NO: 240 - DNA, 15 bp, Artificial Sequence
  Description of Artificial Sequence Synthetic oligonucleotide sequence motif or oligomeric compound
agtcactgtcttcat

SEQ ID NO: 241 - DNA, 16 bp, Artificial Sequence
  Description of Artificial Sequence Synthetic oligonucleotide sequence motif or oligomeric compound
aagtcactgtcttcat

SEQ ID NO: 242 - DNA, 16 bp, Artificial Sequence
  Description of Artificial Sequence Synthetic oligonucleotide sequence motif or oligomeric compound
caaagtcactgtcttc

SEQ ID NO: 243 - DNA, 15 bp, Artificial Sequence
  Description of Artificial Sequence Synthetic oligonucleotide sequence motif or oligomeric compound
caaagtcactgtctt

SEQ ID NO: 244 - DNA, 16 bp, Artificial Sequence
  Description of Artificial Sequence Synthetic oligonucleotide sequence motif or oligomeric compound
ccaaagtcactgtctt

SEQ ID NO: 245 - DNA, 15 bp, Artificial Sequence
  Description of Artificial Sequence Synthetic oligonucleotide sequence motif or oligomeric compound
ccaaagtcactgtct

SEQ ID NO: 246 - DNA, 14 bp, Artificial Sequence
  Description of Artificial Sequence Synthetic oligonucleotide sequence motif or oligomeric compound
ccaaagtcactgtc

SEQ ID NO: 247 - DNA, 12 bp, Artificial Sequence
  Description of Artificial Sequence Synthetic oligonucleotide sequence motif or oligomeric compound
tagcaatctcgc

SEQ ID NO: 248 - DNA, 15 bp, Artificial Sequence
  Description of Artificial Sequence Synthetic oligonucleotide sequence motif or oligomeric compound
agatggcagcagagc

SEQ ID NO: 249 - DNA, 16 bp, Artificial Sequence
  Description of Artificial Sequence Synthetic oligonucleotide sequence motif or oligomeric compound
aagatggcagcagagc

SEQ ID NO: 250 - DNA, 16 bp, Artificial Sequence
  Description of Artificial Sequence Synthetic oligonucleotide sequence motif or oligomeric compound
aaagatggcagcagag

SEQ ID NO: 251 - DNA, 15 bp, Artificial Sequence
  Description of Artificial Sequence Synthetic oligonucleotide sequence motif or oligomeric compound
aaagatggcagcaga

SEQ ID NO: 252 - DNA, 16 bp, Artificial Sequence
  Description of Artificial Sequence Synthetic oligonucleotide sequence motif or oligomeric compound
caaagatggcagcaga

SEQ ID NO: 253 - DNA, 15 bp, Artificial Sequence
  Description of Artificial Sequence Synthetic oligonucleotide sequence motif or oligomeric compound
caaagatggcagcag

SEQ ID NO: 254 - DNA, 16 bp, Artificial Sequence
  Description of Artificial Sequence Synthetic oligonucleotide sequence motif or oligomeric compound
acaaagatggcagcag

SEQ ID NO: 255 - DNA, 14 bp, Artificial Sequence
  Description of Artificial Sequence Synthetic oligonucleotide sequence motif or oligomeric compound
aaactcagaatata

SEQ ID NO: 256 - DNA, 13 bp, Artificial Sequence
  Description of Artificial Sequence Synthetic oligonucleotide sequence motif or oligomeric compound
aaactcagaatat

SEQ ID NO: 257 - DNA, 14 bp, Artificial Sequence
  Description of Artificial Sequence Synthetic oligonucleotide sequence motif or oligomeric compound
tacagcaccacaaa

SEQ ID NO: 258 - DNA, 15 bp, Artificial Sequence
  Description of Artificial Sequence Synthetic oligonucleotide sequence motif or oligomeric compound
ctacagcaccacaaa

SEQ ID NO: 259 - DNA, 14 bp, Artificial Sequence
  Description of Artificial Sequence Synthetic oligonucleotide sequence motif or oligomeric compound
ctacagcaccacaa

SEQ ID NO: 260 - DNA, 13 bp, Artificial Sequence
  Description of Artificial Sequence Synthetic oligonucleotide sequence motif or oligomeric compound
ctacagcaccaca

SEQ ID NO: 261 - DNA, 14 bp, Artificial Sequence
  Description of Artificial Sequence Synthetic oligonucleotide sequence motif or oligomeric compound
gtccatcacagtaa

SEQ ID NO: 262 - DNA, 15 bp, Artificial Sequence
  Description of Artificial Sequence Synthetic oligonucleotide sequence motif or oligomeric compound
tgtccatcacagtaa

SEQ ID NO: 263 - DNA, 6390 bp, Homo sapiens
gtgcgccactgcagctggcattggccgggactggaagtgcgggcttctgcagcagccgaagctggagctgctaggcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacatacaaaaaaaaaaaaaaaaaaa

SEQ ID NO: 264 - DNA, 14 bp, Artificial Sequence
  Description of Artificial Sequence Synthetic oligonucleotide sequence motif or oligomeric compound
ggcagataagaaac

SEQ ID NO: 265 - DNA, 34 bp, Artificial Sequence
  Description of Artificial Sequence Synthetic oligonucleotide sequence motif or oligomeric compound
gtttcattgatataaataacattccgcaaaccca

SEQ ID NO: 266 - DNA, 16 bp, Artificial Sequence
  Description of Artificial Sequence Synthetic oligonucleotide sequence motif or oligomeric compound
caaacatgcccttatg

SEQ ID NO: 267 - DNA, 13 bp, Artificial Sequence
  Description of Artificial Sequence Synthetic oligonucleotide sequence motif or oligomeric compound
caattgcctcttg

SEQ ID NO: 268 - DNA, 14 bp, Artificial Sequence
  Description of Artificial Sequence Synthetic oligonucleotide sequence motif or oligomeric compound
aagaagctgttgaa

SEQ ID NO: 269 - DNA, 16 bp, Artificial Sequence
  Description of Artificial Sequence Synthetic oligonucleotide sequence motif or oligomeric compound
ttcgtctcagttgcag

SEQ ID NO: 270 - DNA, 20 bp, Artificial Sequence
  Description of Artificial Sequence Synthetic oligonucleotide sequence motif or oligomeric compound
gggatgttgagattattgcc

SEQ ID NO: 271 - DNA, 14 bp, Artificial Sequence
  Description of Artificial Sequence Synthetic oligonucleotide sequence motif or oligomeric compound
gtttcatcgagcct

SEQ ID NO: 272 - DNA, 18 bp, Artificial Sequence
  Description of Artificial Sequence Synthetic oligonucleotide sequence motif or oligomeric compound
attgtagtgaccttcgat

SEQ ID NO: 273 - DNA, 15 bp, Artificial Sequence
  Description of Artificial Sequence Synthetic oligonucleotide sequence motif or oligomeric compound
ttctaaatattcctt

SEQ ID NO: 274 - DNA, 28 bp, Artificial Sequence
  Description of Artificial Sequence Synthetic oligonucleotide sequence motif or oligomeric compound
attccctgcctgtgtctgtagaggagca

SEQ ID NO: 275 - DNA, 18 bp, Artificial Sequence
  Description of Artificial Sequence Synthetic oligonucleotide sequence motif or oligomeric compound
ttcatcacaaagaagtct

SEQ ID NO: 276 - DNA, 14 bp, Artificial Sequence
  Description of Artificial Sequence Synthetic oligonucleotide sequence motif or oligomeric compound
acatcttctgaatt

SEQ ID NO: 277 - DNA, 19 bp, Artificial Sequence
  Description of Artificial Sequence Synthetic oligonucleotide sequence motif or oligomeric compound
gtgtgggtgattgtgacac

SEQ ID NO: 278 - DNA, 13 bp, Artificial Sequence
  Description of Artificial Sequence Synthetic oligonucleotide sequence motif or oligomeric compound
gggacagttgtgc

SEQ ID NO: 279 - DNA, 18 bp, Artificial Sequence
  Description of Artificial Sequence Synthetic oligonucleotide sequence motif or oligomeric compound
gctgtagaagttgagttc

SEQ ID NO: 280 - DNA, 16 bp, Artificial Sequence
  Description of Artificial Sequence Synthetic oligonucleotide sequence motif or oligomeric compound
ctgcaagctatgatgg

SEQ ID NO: 281 - DNA, 14 bp, Artificial Sequence
  Description of Artificial Sequence Synthetic oligonucleotide sequence motif or oligomeric compound
ctcctggctgatca

SEQ ID NO: 282 - DNA, 19 bp, Artificial Sequence
  Description of Artificial Sequence Synthetic oligonucleotide sequence motif or oligomeric compound
gagagaaggtagcacaggc

SEQ ID NO: 283 - DNA, 15 bp, Artificial Sequence
  Description of Artificial Sequence Synthetic oligonucleotide sequence motif or oligomeric compound
agaatgccatactgg

SEQ ID NO: 284 - DNA, 15 bp, Artificial Sequence
  Description of Artificial Sequence Synthetic oligonucleotide sequence motif or oligomeric compound
atcttcctggtcatc

SEQ ID NO: 285 - DNA, 21 bp, Artificial Sequence
  Description of Artificial Sequence Synthetic oligonucleotide sequence motif or oligomeric compound
aagcttcttgtcccactgctg

SEQ ID NO: 286 - DNA, 23 bp, Artificial Sequence
  Description of Artificial Sequence Synthetic oligonucleotide sequence motif or oligomeric compound
ccaaagtcactgtcttcatcttc

SEQ ID NO: 287 - DNA, 12 bp, Artificial Sequence
  Description of Artificial Sequence Synthetic oligonucleotide sequence motif or oligomeric compound
tagcaatctcgc

SEQ ID NO: 288 - DNA, 19 bp, Artificial Sequence
  Description of Artificial Sequence Synthetic oligonucleotide sequence motif or oligomeric compound
acaaagatggcagcagagc

SEQ ID NO: 289 - DNA, 27 bp, Artificial Sequence
  Description of Artificial Sequence Synthetic oligonucleotide sequence motif or oligomeric compound
ctacagcaccacaaaactcagaatata

SEQ ID NO: 290 - DNA, 15 bp, Artificial Sequence
  Description of Artificial Sequence Synthetic oligonucleotide sequence motif or oligomeric compound
tgtccatcacagtaa

SEQ ID NO: 291 - DNA, 14 bp, Artificial Sequence
  Description of Artificial Sequence Synthetic oligonucleotide sequence motif or oligomeric compound
ggcagataagaaac

SEQ ID NO: 292 - DNA, 12 bp, Artificial Sequence
  Description of Artificial Sequence Synthetic oligonucleotide sequence motif or oligomeric compound
ataacattccgc

SEQ ID NO: 293 - DNA, 14 bp, Artificial Sequence
  Description of Artificial Sequence Synthetic oligonucleotide sequence motif or oligomeric compound
tgtagtgaccttcg

SEQ ID NO: 294 - DNA, 16 bp, Artificial Sequence
  Description of Artificial Sequence Synthetic oligonucleotide sequence motif or oligomeric compound
ttccctgcctgtgtct

SEQ ID NO: 295 - DNA, 14 bp, Artificial Sequence
  Description of Artificial Sequence Synthetic oligonucleotide sequence motif or oligomeric compound
attccctgcctgtg

SEQ ID NO: 296 - DNA, 15 bp, Artificial Sequence
  Description of Artificial Sequence Synthetic oligonucleotide sequence motif or oligomeric compound
gtgggtgattgtgac

SEQ ID NO: 297 - DNA, 15 bp, Artificial Sequence
  Description of Artificial Sequence Synthetic oligonucleotide sequence motif or oligomeric compound
gtgtgggtgattgtg

SEQ ID NO: 298 - DNA, 15 bp, Artificial Sequence
  Description of Artificial Sequence Synthetic oligonucleotide sequence motif or oligomeric compound
tgtagaagttgagtt

SEQ ID NO: 299 - DNA, 15 bp, Artificial Sequence
  Description of Artificial Sequence Synthetic oligonucleotide sequence motif or oligomeric compound
gagagaaggtagcac

SEQ ID NO: 300 - DNA, 16 bp, Artificial Sequence
  Description of Artificial Sequence Synthetic oligonucleotide sequence motif or oligomeric compound
aagatggcagcagagc

SEQ ID NO: 301 - DNA, 16 bp, Artificial Sequence
  Description of Artificial Sequence Synthetic oligonucleotide sequence motif or oligomeric compound
aaagatggcagcagag

SEQ ID NO: 302 - DNA, 16 bp, Artificial Sequence
  Description of Artificial Sequence Synthetic oligonucleotide sequence motif or oligomeric compound
acaaagatggcagcag

(19) United States
(12) Patent Application Publication
(10) Pub. No.: US 2012/0004276 A1
(43) Pub. Date: Jan. 5, 2012

(54) RNA ANTAGONIST COMPOUNDS FOR THE INHIBITION OF EXPRESSION OF MITOCHONDRIAL GLYCEROL-3 PHOSPHATE ACYLTRANSFERASE 1 (MTGPAT1)

(76) Inventors: Marie Lindholm, Malmo (SE); Ellen Marie Straarup, Bistrup (DK)

(21) Appl. No.: 13/000,974

(22) PCT Filed: Jun. 24, 2009
(86) PCT No.: PCT/EP09/57907

§ 371 Date: Sep. 12, 2011

Related U.S. Application Data
(60) Provisional application No. 61/077,942, filed on Jul. 3, 2008.

(30) Foreign Application Priority Data
Jul. 3, 2008 (EP) 08104623.7

(51) Int. Cl. A61K 31/7088; A61P 3/10; A61P 3/04; A61P 1/16; C07H 21/04; A61P 3/00

(52) U.S. Cl. 514/44 A; 536/24.5

(57) ABSTRACT

The present invention relates to oligomer compounds (oligomers), which target mtGPAT1mRNA in a cell, leading to reduced expression of mtGPAT1. Reduction of mtGPAT1 expression is beneficial for the treatment of certain medical disorders, such as overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).

### FIELD OF INVENTION

The present invention relates to oligomeric compounds (oligomers), that target mtGPAT1 mRNA in a cell, leading to reduced expression of mtGPAT1. Reduction of mtGPAT1 expression is beneficial for a range of medical disorders, such as overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).

### BACKGROUND

Mitochondrial glycerol-3-phosphate acyltransferase 1 (EC 2.3.1.15, also known as GPAT1, mtGPAT1, GPAM, mtGPAM) play a major role in hepatic triglyceride formation, where high levels of mtGPAT1 activity results in fatty liver (hepatosteatosis) whereas absence of mtGPAT1 results in low levels of liver triglycerides and stimulated fatty acid oxidation.

The glycerol-3-phosphate acyltransferases (GPATs) is a family of enzymes that catalyze a rate-limiting step in triglyceride synthesis. The enzymes catalyze the formation of an ester bond between glycerol-3-phosphate and an activated fatty acid (acyl-coenzyme A, acyl-CoA). It was early recognized that more than one enzyme was responsible for GPAT enzymatic activity in cells, with enzymatic activity present in the outer membrane of both endoplasmatic reticulum and mitochondria, and with one fraction of enzymes insensitive respective sensitive to inactivation by NEM (Coleman et al. (2000) Annu. Rev. Nutr. 20, 77-103-3; Coleman et al. (2004) Prog. Lipid Res. 43, 134-176; Coleman (2007) Cell Metab 5, 87-89).

MtGPAT1 has been identified as a GPAT enzyme insensitive to inactivation by NEM and present in mitochondria only. Activity of mtGPAT1 is low in extrahepatic tissues where it is responsible for 10% of total GPAT activity, whereas the activity is high in liver where mtGPAT1 accounts for up to 50% of total GPAT activity (Coleman et al. (2000) Annu. Rev. Nutr. 20, 77-103-3; Coleman et al. (2004) Prog. Lipid Res. 43, 134-176; Coleman (2007) Cell Metab 5, 87-89). Lysophosphatidic acid (LPA), the product of all GPAT activity, can proceed towards synthesis of both triglycerides and phospholipids. Most enzymes involved in triglyceride synthesis are present in the endoplasmatic reticulum, and mtGPAT1 was therefore earlier believed to be involved mainly in phospholipid precursor synthesis. However, hormonal and nutritional regulation of mtGPAT1 activity indicates a critical role in hepatic triglyceride synthesis (Coleman et al. (2000) Annu. Rev. Nutr. 20, 77-103-3; Coleman et al. (2004) Prog. Lipid Res. 43, 134-176; Coleman (2007) Cell Metab 5, 87-89). MtGPAT1 activity is dramatically up-regulated in response to feeding and in obese mice (Xu et al. Biochem. Biophys. Res. Commun. 349, 439-448).

Over-expression of mtGPAT1 in CHO or HEK293 cells results in a solid increase in levels of intracellular triglyceride (Igal et al. (2001) J. Biol. Chem. 276, 42205-42212). Over-expression of mtGPAT1 in liver cells results in an even higher level of intracellular lipid accumulation (Lewin et al. (2005) Am. J. Physiol Endocrinol. Metab 288, E835-E844) concomitant with a decrease in utilization of fatty acids for cellular fuel (β-oxidation)—it appears as if high levels of mtGPAT1 activity results in increased hepatic fatty acid uptake and triglyceride synthesis (lipogenic anabolism) and decreased fatty acid oxidation (lipid catabolism). This is in agreement with a view of a malonyl-CoA controlled “metabolic switch”, where the energy requirement of cells (through control of malonyl-CoA concentrations) steers activated fatty acids towards lipogenesis/storage (mtGPAT1 activity) or transfer into mitochondria followed by fatty acid oxidation (with carnitin palmitoyl transferase-1, CPT-1, as rate limiting enzyme). In all cells expressing high levels of mtGPAT1 triglyceride synthesis was favored over insertion of activated fatty acids in phospholipids or cholesterol ester.

Transient hepatic adenovirus-induced over expression of mtGPAT1 results in a massive increase in liver triacylglycerol, i.e. hepatosteatosis (Linden et al. (2006) FASEB J. 20, 434-443) and insulin resistance (Nagle et al. (2007) J. Biol. Chem. 282, 14807-14815). MtGPAT1 knockout mice have been generated. When kept on standard chow animals have lower weight and gonadal fat pad weight, lower liver triglyceride levels, lower plasma triglyceride, and lower VLDL secretion (Hammond et al. (2002) Mol. Cell Biol. 22, 8204-8214; Yazdi et al. (2008) Biochem. Biophys. Res. Commun. 369, 1065-1070). MtGPAT1 knock out animals also appears to be protected against insulin resistance (Neschen et al. (2005) Cell Metab 2, 55-65). In mice kept on a high fat, high sucrose diet for 4 months absence of mtGPAT1 resulted in a 60% decrease in hepatic triglyceride content, together with indications of stimulated fatty acid oxidation such as increased levels of plasma β-hydroxybutyrate (Hammond et al. (2005) J. Biol. Chem. 280, 25629-25636). Old mtGPAT1 knockout mice have increased hepatic accumulation of long chain fatty acid-CoA, suggesting that a balanced down-regulation of enzymatic activity is preferable compared to complete absence of the protein (Hammond et al. (2005) J. Biol. Chem. 280, 25629-25636). However, absence of mtGPAT1 does not appear to result in any gross changes in liver size, liver cell number, or mitochondrial morphology (Hammond et al. (2007) Exp. Mol. Pathol. 82, 210-219). The overall conclusion is that high mtGPAT1 activity is correlated to obesity, insulin resistance, and hepatic lipid accumulation.

Inhibition of mtGPAT1 activity has so far been limited to small molecules directed towards the active site of the enzyme. However, there is a large degree of homology of protein sequence at the active site of different members of the GPAT family (Gonzalez-Baro et al. (2007) Am. J. Physiol Gastrointest. Liver Physiol 292, G1195-G1199) making design of small molecule inhibitors specific for one singular member of the protein family a challenge.

Thus, there is a need for subtype specific GPAT inhibitors, such as mtGPAT1 specific inhibitors. The LNA containing RNA antagonists of the present invention are such mtGPAT1 specific inhibitors that meet the unmet need for therapeutic, diagnostic and research applications involving modulation of mtGPAT1 expression.

### SUMMARY OF INVENTION

The invention provides an oligomer of between 10-30 nucleotides in length which comprises a contiguous nucleotide sequence of a total of between 10-30 nucleotides, wherein said contiguous nucleotide sequence is at least 80% (e.g., 85%, 90%, 95%, 98%, or 99%) homologous to a region corresponding to the reverse complement of a mammalian mtGPAT1 gene or mRNA, such as SEQ ID NO: 263 or naturally occurring variant thereof. Thus, for example, the oligomer hybridizes to a single stranded nucleic acid molecule having the sequence of a portion of SEQ ID NO: 263.

The invention provides for a conjugate comprising the oligomer according to the invention, and at least one non-nucleotide or non-polynucleotide moiety covalently attached to said oligomer.

The invention provides for a pharmaceutical composition comprising the oligomer or the conjugate according to the invention, and a pharmaceutically acceptable diluent, carrier, salt or adjuvant.

The invention provides for the oligomer or the conjugate according to invention, for use as a medicament, such as for the treatment of overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).

The invention provides for the use of an oligomer or the conjugate according to the invention, for the manufacture of a medicament for the treatment of overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).

The invention provides for a method of treating overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM), said method comprising administering an oligomer, a conjugate or a pharmaceutical composition according to the invention, to a patient suffering from, or likely to suffer from overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).

The invention provides for a method for the inhibition of mtGPAT1 in a cell which is expressing mtGPAT1, said method comprising administering an oligomer, or a conjugate according to the invention to said cell so as to effect the inhibition of mtGPAT1 in said cell.

### BRIEF DESCRIPTION OF FIGURES

FIG. 1: demonstrates that a range of LNA containing single stranded antisense oligonucleotides directed against mtGPAT1 are potent in the same nanomolar range in vitro. A) Relative mtGPAT1 mRNA expression in LTK-D2 cells after lipid-assisted transfection with a series of LNA containing antisense molecules directed against mtGPAT1. Data represents mean±SD for mtGPAT1/GADPH mRNA expression expressed as percent of corresponding mRNA ratio in mock transfected cells. B) Relative mtGPAT1 mRNA expression in HuH7 cells after lipid-assisted transfection with a series of LNA containing antisense molecules directed against mtGPAT1. Data represents mean±SD for mtGPAT1/GADPH mRNA expression expressed as percent of corresponding mRNA ratio in mock transfected cells.

FIG. 2: In vivo downregulation of liver mtGPAT mRNA expression in female C57BL/6 mice. The effect of 5 different mtGPAT antisense oligomers, SEQ ID # 33, 125, 147, 176, and 249 on liver mtGPAT mRNA expression was tested.

### BRIEF DESCRIPTION OF SEQUENCE ID'S

1-262 are presented in Table 1
263 is presented in the sequence list after the examples section
264-290 are presented in Table 2
A list of specially preferred antisense sequences selected from those of Table 1, are presented in Table 3.

### DETAILED DESCRIPTION OF INVENTION

### The Oligomer

The present invention employs oligomeric compounds (referred herein as oligomers), for use in modulating the function of nucleic acid molecules encoding mammalian mtGPAT1, such as the mtGPAT1 nucleic acid shown in SEQ ID 263, and naturally occurring variants of such nucleic acid molecules encoding mammalian mtGPAT1. The term “oligomer” in the context of the present invention, refers to a molecule formed by covalent linkage of two or more nucleotides (i.e. an oligonucleotide). The oligomer consists or comprises of a contiguous nucleotide sequence of between 10-30 nucleotides in length.

In various embodiments, the compound of the invention does not comprise RNA (units). It is preferred that the compound according to the invention is a linear molecule or is synthesised as a linear molecule. The oligomer is a single stranded molecule, and preferably does not comprise short regions of, for example, at least 3, 4 or 5 contiguous nucleotides, which are complementary to equivalent regions within the same oligomer (i.e. duplexes)—in this regards, the oligomer is not (essentially) double stranded. In some embodiments, the oligomer is essentially not double stranded, such as is not a siRNA. In various embodiments, the oligomer of the invention may consist entirely of the contiguous nucleotide region. Thus, the oligomer is not substantially selfcomplementary.

### The Target

Suitably the oligomer of the invention is capable of down-regulating expression of the mtGPAT1 gene. In this regards, the oligomer of the invention can effect the inhibition of mtGPAT1, typically in a mammalian such as a human cell. In some embodiments, the oligomers of the invention bind to the target nucleic acid and effect inhibition of expression of at least 10% or 20% compared to the normal expression level, more preferably at least a 30%, 40%, 50%, 60%, 70%, 80%, 90% or 95% inhibition compared to the normal expression level. In some embodiments, such modulation is seen when using between 0.04 and 25 nM, such as between 0.8 and 20 nM concentration of the compound of the invention. In the same or a different embodiment, the inhibition of expression is less than 100%, such as less than 98% inhibition, less than 95% inhibition, less than 90% inhibition, less than 80% inhibition, such as less than 70% inhibition. Modulation of expression level may be determined by measuring protein levels, e.g. by the methods such as SDS-PAGE followed by western blotting using suitable antibodies raised against the target protein. Alternatively, modulation of expression levels can be determined by measuring levels of mRNA, e.g. by northern blotting or quantitative RT-PCR. When measuring via mRNA levels, the level of down-regulation when using an appropriate dosage, such as between 0.04 and 25 nM, such as between 0.8 and 20 nM concentration, is, In some embodiments, typically to a level of between 10-20% the normal levels in the absence of the compound of the invention.

The invention therefore provides a method of down-regulating or inhibiting the expression of mtGPAT1 protein and/or mRNA in a cell which is expressing mtGPAT1 protein and/or mRNA, said method comprising administering the oligomer or conjugate according to the invention to said cell to down-regulating or inhibiting the expression of mtGPAT1 protein and/or mRNA in said cell. Suitably the cell is a mammalian cell such as a human cell. The administration may occur, In some embodiments, in vitro. The administration may occur, In some embodiments, in vivo.

The term “target nucleic acid”, as used herein refers to the DNA or RNA encoding mammalian mtGPAT1 polypeptide, such as human mtGPAT1, such as SEQ ID NO: 263. mtGPAT1 encoding nucleic acids or naturally occurring variants thereof, and RNA nucleic acids derived therefrom, preferably mRNA, such as pre-mRNA, although preferably mature mRNA. In some embodiments, for example when used in research or diagnostics the “target nucleic acid” may be a cDNA or a synthetic oligonucleotide derived from the above DNA or RNA nucleic acid targets. The oligomer according to the invention is preferably capable of hybridising to the target nucleic acid. It will be recognised that SEQ ID NO: 263 is a cDNA sequences, and as such, corresponds to the mature mRNA target sequence, although uracil is replaced with thymidine in the cDNA sequences.

The term “naturally occurring variant thereof” refers to variants of the mtGPAT1 polypeptide of nucleic acid sequence which exist naturally within the defined taxonomic group, such as mammalian, such as mouse, monkey, and preferably human. Typically, when referring to “naturally occurring variants” of a polynucleotide the term also may encompass any allelic variant of the mtGPAT1 encoding genomic DNA which are found at the Chromosome 10; Location: 10q25.2 Mb by chromosomal translocation or duplication, and the RNA, such as mRNA derived therefrom. “Naturally occurring variants” may also include variants derived from alternative splicing of the mtGPAT1 mRNA. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

### Sequences

The oligomers comprise or consist of a contiguous nucleotide sequence which corresponds to the reverse complement of a nucleotide sequence present in SEQ ID NO: 263. Thus, the oligomer can comprise or consist of, or a sequence selected from the group consisting of SEQ ID NOS: 1-262, wherein said oligomer (or contiguous nucleotide portion thereof) may optionally have one, two, or three mismatches against said selected sequence.

TABLE 1
List of oligomeric sequences of the invention,
The oligomeric sequences in this table may be designed
according to the invention, as described elsewhere, by
including nucleotide analogues that increase the Tm of the
oligonucleotide. Further, phosphorothioate linkages may be
present as internucleotide bonds.

| | Start point | | |
|---|---|---|---|
| | of target | | |
| | sequence in | | |
| Test | human mRNA | | Type of |
| substance | as of | | oligonucleotide |
| Seq. ID # | SEQ ID 263 | Oligonucleotide sequence | sequence |
| 1 | 232 | 5′-GCAGATAAGAAAC-3′ | antisense sequence |
| 2 | 232 | 5′- [symbol] [symbol] AₛGₛAₛTₛAₛAₛGₛAₛ | LNA anitsense |
| | | [symbol] -3′ | oligonucleotide |
| 3 | 452 | 5′-TTCCGCAAACCCA-3′ | antisense sequence |
| 4 | 452 | 5′-ATTCCGCAAACCCA-3′ | antisense sequence |
| 5 | 452 | 5′-CATTCCGCAAACCCA-3′ | antisense sequence |
| 6 | 452 | 5′-ACATTCCGCAAACCCA-3′ | antisense sequence |
| 7 | 453 | 5′-TTCCGCAAACCC-3′ | antisense sequence |
| 8 | 453 | 5′-ATTCCGCAAACCC-3′ | antisense sequence |
| 9 | 453 | 5′-CATTCCGCAAACCC-3′ | antisense sequence |
| 10 | 453 | 5′-ACATTCCGCAAACCC-3′ | antisense sequence |
| 11 | 453 | 5′-AACATTCCGCAAACCC-3′ | antisense sequence |
| 12 | 454 | 5′-ACATTCCGCAAACC-3 ′ | antisense sequence |
| 13 | 454 | 5′-AACATTCCGCAAACC-3′ | antisense sequence |
| 14 | 454 | 5′-TAACATTCCGCAAACC-3′ | antisense sequence |
| 15 | 455 | 5′-ACATTCCGCAAAC-3′ | antisense sequence |
| 16 | 455 | 5′-AACATTCCGCAAAC-3′ | antisense sequence |
| 17 | 455 | 5′-TAACATTCCGCAAAC-3′ | antisense sequence |
| 18 | 455 | 5′-ATAACATTCCGCAAAC-3′ | antisense sequence |
| 19 | 456 | 5′-AACATTCCGCAAA-3′ | antisense sequence |
| 20 | 456 | 5′-TAACATTCCGCAAA-3′ | antisense sequence |
| 21 | 456 | 5′-ATAACATTCCGCAAA-3′ | antisense sequence |
| 22 | 456 | 5′-AATAACATTCCGCAAA-3′ | antisense sequence |
| 23 | 457 | 5′-AACATTCCGCAA-3′ | antisense sequence |
| 24 | 457 | 5′-TAACATTCCGCAA-3′ | antisense sequence |
| 25 | 457 | 5′-ATAACATTCCGCAA-3′ | antisense sequence |
| 26 | 457 | 5′-AATAACATTCCGCAA-3′ | antisense sequence |
| 27 | 457 | 5′-AAATAACATTCCGCAA-3′ | antisense sequence |
| 28 | 458 | 5′-TAACATTCCGCA-3′ | antisense sequence |
| 29 | 458 | 5′-ATAACATTCCGCA-3′ | antisense sequence |
| 30 | 458 | 5′-AATAACATTCCGCA-3′ | antisense sequence |
| 31 | 458 | 5′-AAATAACATTCCGCA-3′ | antisense sequence |
| 32 | 458 | 5′-TAAATAACATTCCGCA-3′ | antisense sequence |
| 33 | 459 | 5′- [symbol] AₛAₛCₛAₛTₛTₛCₛCₛ [symbol] -3′ | LNA antisense |
| | | | oligonucleotide |
| 34 | 459 | 5′-AATAACATTCCGC-3′ | antisense sequence |
| 35 | 459 | 5′-AAATAACATTCCGC-3′ | antisense sequence |
| 36 | 459 | 5′-TAAATAACATTCCGC-3′ | antisense sequence |
| 37 | 459 | 5′-ATAAATAACATTCCGC-3′ | antisense sequence |
| 38 | 460 | 5′-AATAACATTCCG-3′ | antisense sequence |
| 39 | 460 | 5′-AAATAACATTCCG-3′ | antisense sequence |
| 40 | 460 | 5′-TAAATAACATTCCG-3′ | antisense sequence |
| 41 | 460 | 5′-ATAAATAACATTCCG-3′ | antisense sequence |
| 42 | 460 | 5′-TATAAATAACATTCCG-3′ | antisense sequence |
| 43 | 461 | 5′-TATAAATAACATTCC-3′ | antisense sequence |
| 44 | 461 | 5′-ATATAAATAACATTCC-3′ | antisense sequence |
| 45 | 462 | 5′-TATAAATAACATTC-3′ | antisense sequence |
| 46 | 462 | 5′-ATATAAATAACATTC-3′ | antisense sequence |
| 47 | 462 | 5′-GATATAAATAACATTC-3′ | antisense sequence |
| 48 | 463 | 5′-GATATAAATAACATT-3′ | antisense sequence |
| 49 | 463 | 5′-TGATATAAATAACATT-3′ | antisense sequence |
| 50 | 464 | 5′-TGATATAAATAACAT-3′ | antisense sequence |
| 51 | 464 | 5′-TTGATATAAATAACAT-3′ | antisense sequence |
| 52 | 465 | 5′-TGATATAAATAACA-3′ | antisense sequence |
| 53 | 465 | 5′-TTGATATAAATAACA-3′ | antisense sequence |
| 54 | 465 | 5′-ATTGATATAAATAACA-3′ | antisense sequence |
| 55 | 466 | 5′-TGATATAAATAAC-3′ | antisense sequence |
| 56 | 466 | 5′-TTGATATAAATAAC-3′ | antisense sequence |
| 57 | 466 | 5′-ATTGATATAAATAAC-3′ | antisense sequence |
| 58 | 466 | 5′-CATTGATATAAATAAC-3′ | antisense sequence |
| 59 | 467 | 5′-ATTGATATAAATAA-3′ | antisense sequence |
| 60 | 467 | 5′-CATTGATATAAATAA-3′ | antisense sequence |
| 61 | 467 | 5′-TCATTGATATAAATAA-3′ | antisense sequence |
| 62 | 470 | 5′-GTTTCATTGATATAAA-3′ | antisense sequence |
| 63 | 471 | 5′-GTTTCATTGATATAA-3′ | antisense sequence |
| 64 | 472 | 5′-GTTTCATTGATATA-3′ | antisense sequence |
| 65 | 556 | 5′-ACATGCCCTTATG-3′ | antisense sequence |
| 66 | 556 | 5′-AACATGCCCTTATG-3′ | antisense sequence |
| 67 | 556 | 5′-AAACATGCCCTTATG-3′ | antisense sequence |
| 68 | 556 | 5′-CAAACATGCCCTTATG-3′ | antisense sequence |
| 69 | 557 | 5′-AACATGCCCTTAT-3′ | antisense sequence |
| 70 | 557 | 5′-AAACATGCCCTTAT-3′ | antisense sequence |
| 71 | 557 | 5′-CAAACATGCCCTTAT-3′ | antisense sequence |
| 72 | 558 | 5′-CAAACATGCCCTTA-3′ | antisense sequence |
| 73 | 559 | 5′-CAAACATGCCCTT-3′ | antisense sequence |
| 74 | 613 | 5′-CAATTGCCTCTTG-3′ | antisense sequence |
| 75 | 784 | 5′-AGAAGCTGTTGAA-3′ | antisense sequence |
| 76 | 784 | 5′-AAGAAGCTGTTGAA-3′ | antisense sequence |
| 77 | 842 | 5′-CGTCTCAGTTGCAG-3′ | antisense sequence |
| 78 | 842 | 5′-TCGTCTCAGTTGCAG-3′ | antisense sequence |
| 79 | 842 | 5′-TTCGTCTCAGTTGCAG-3′ | antisense sequence |
| 80 | 843 | 5′-CGTCTCAGTTGCA-3′ | antisense sequence |
| 81 | 843 | 5′-TCGTCTCAGTTGCA-3′ | antisense sequence |
| 82 | 843 | 5′-TTCGTCTCAGTTGCA-3′ | antisense sequence |
| 83 | 844 | 5′-TCGTCTCAGTTGC-3′ | antisense sequence |
| 84 | 844 | 5′-TTCGTCTCAGTTGC-3′ | antisense sequence |
| 85 | 845 | 5′-TCGTCTCAGTTG-3′ | antisense sequence |
| 86 | 845 | 5′-TTCGTCTCAGTTG-3′ | antisense sequence |
| 87 | 846 | 5′-TTCGTCTCAGTT-3′ | antisense sequence |
| 88 | 961 | 5′-TGAGATTATTGCC-3′ | antisense sequence |
| 89 | 961 | 5′-TTGAGATTATTGCC-3′ | antisense sequence |
| 90 | 961 | 5′-GTTGAGATTATTGCC-3′ | antisense sequence |
| 91 | 961 | 5′-TGTTGAGATTATTGCC-3′ | antisense sequence |
| 92 | 962 | 5′-GTTGAGATTATTGC-3′ | antisense sequence |
| 93 | 962 | 5′-TGTTGAGATTATTGC-3′ | antisense sequence |
| 94 | 962 | 5′-ATGTTGAGATTATTGC-3′ | antisense sequence |
| 95 | 963 | 5′-GTTGAGATTATTG-3′ | antisense sequence |
| 96 | 963 | 5′-TGTTGAGATTATTG-3′ | antisense sequence |
| 97 | 963 | 5′-ATGTTGAGATTATTG-3′ | antisense sequence |
| 98 | 963 | 5′-GATGTTGAGATTATTG-3′ | antisense sequence |
| 99 | 964 | 5′-ATGTTGAGATTATT-3′ | antisense sequence |
| 100 | 964 | 5′-GATGTTGAGATTATT-3′ | antisense sequence |
| 101 | 964 | 5′-GGATGTTGAGATTATT-3′ | antisense sequence |
| 102 | 965 | 5′-ATGTTGAGATTAT-3′ | antisense sequence |
| 103 | 965 | 5′-GATGTTGAGATTAT-3′ | antisense sequence |
| 104 | 965 | 5′-GGATGTTGAGATTAT-3′ | antisense sequence |
| 105 | 965 | 5′-GGGATGTTGAGATTAT-3′ | antisense sequence |
| 106 | 966 | 5′-GATGTTGAGATTA-3′ | antisense sequence |
| 107 | 966 | 5′-GGATGTTGAGATTA-3′ | antisense sequence |
| 108 | 966 | 5′-GGGATGTTGAGATTA-3′ | antisense sequence |
| 109 | 967 | 5′-GGGATGTTGAGATT-3′ | antisense sequence |
| 110 | 1030 | 5′-TTCATCGAGCCT-3′ | antisense sequence |
| 111 | 1030 | 5′-TTTCATCGAGCCT-3′ | antisense sequence |
| 112 | 1030 | 5′-GTTTCATCGAGCCT-3′ | antisense sequence |
| 113 | 1031 | 5′-GTTTCATCGAGCC-3′ | antisense sequence |
| 114 | 1032 | 5′-GTTTCATCGAGC-3′ | antisense sequence |
| 115 | 1273 | 5′-AGTGACCTTCGAT-3′ | antisense sequence |
| 116 | 1273 | 5′-TAGTGACCTTCGAT-3′ | antisense sequence |
| 117 | 1273 | 5′-GTAGTGACCTTCGAT-3′ | antisense sequence |
| 118 | 1273 | 5′-TGTAGTGACCTTCGAT-3′ | antisense sequence |
| 119 | 1274 | 5′-TAGTGACCTTCGA-3′ | antisense sequence |
| 120 | 1274 | 5′-GTAGTGACCTTCGA-3′ | antisense sequence |
| 121 | 1274 | 5′-TGTAGTGACCTTCGA-3′ | antisense sequence |
| 122 | 1274 | 5′-TTGTAGTGACCTTCGA-3′ | antisense sequence |
| 123 | 1275 | 5′-TAGTGACCTTCG-3′ | antisense sequence |
| 124 | 1275 | 5′-GTAGTGACCTTCG-3′ | antisense sequence |
| 125 | 1275 | 5′- [symbol] ^{o}AₛGₛTₛGₛAₛCₛCₛTₛ [symbol] | LNA antisense |
| | | [symbol] -3′ | oligonucleotide |
| 126 | 1275 | 5′-TTGTAGTGACCTTCG-3′ | antisense sequence |
| 127 | 1275 | 5′-ATTGTAGTGACCTTCG-3′ | antisense sequence |
| 128 | 1276 | 5′-TTGTAGTGACCTTC-3′ | antisense sequence |
| 129 | 1276 | 5′-ATTGTAGTGACCTTC-3′ | antisense sequence |
| 130 | 1277 | 5′-ATTGTAGTGACCTT-3′ | antisense sequence |
| 131 | 1414 | 5′-TTCTAAATATTCCTT-3′ | antisense sequence |
| 132 | 1415 | 5′-TTCTAAATATTCCT-3′ | antisense sequence |
| 133 | 1667 | 5′-CTGTAGAGGAGCA-3′ | antisense sequence |
| 134 | 1674 | 5′-GCCTGTGTCTGTAG-3′ | antisense sequence |
| 135 | 1674 | 5′-TGCCTGTGTCTGTAG-3′ | antisense sequence |
| 136 | 1675 | 5′-TGCCTGTGTCTGTA-3′ | antisense sequence |
| 137 | 1675 | 5′-CTGCCTGTGTCTGTA-3′ | antisense sequence |
| 138 | 1675 | 5′-CCTGCCTGTGTCTGTA-3′ | antisense sequence |
| 139 | 1676 | 5′-CCCTGCCTGTGTCTGT-3′ | antisense sequence |
| 140 | 1677 | 5′-CCCTGCCTGTGTCTG-3′ | antisense sequence |
| 141 | 1677 | 5′-TCCCTGCCTGTGTCTG-3′ | antisense sequence |
| 142 | 1678 | 5′- [symbol] [symbol] CₛCₛTₛGₛCₛCₛTₛGₛTₛGₛ | LNA antisense |
| | | [symbol] -3′ | oligonucleotide |
| 143 | 1679 | 5′-TTCCCTGCCTGTGTC-3′ | antisense sequence |
| 144 | 1679 | 5′-ATTCCCTGCCTGTGTC-3′ | antisense sequence |
| 145 | 1680 | 5′-TTCCCTGCCTGTGT-3′ | antisense sequence |
| 146 | 1680 | 5′-ATTCCCTGCCTGTGT-3′ | antisense sequence |
| 147 | 1681 | 5′- [symbol] ^{o}CₛCₛCₛTₛGₛCₛCₛTₛ [symbol] | LNA antisense |
| | | [symbol] -3′ | oligonucleotide |
| 148 | 1716 | 5′-TCACAAAGAAGTCT-3′ | antisense sequence |
| 149 | 1716 | 5′-ATCACAAAGAAGTCT-3′ | antisense sequence |
| 150 | 1716 | 5′-CATCACAAAGAAGTCT-3′ | antisense sequence |
| 151 | 1717 | 5′-ATCACAAAGAAGTC-3′ | antisense sequence |
| 152 | 1717 | 5′-CATCACAAAGAAGTC-3′ | antisense sequence |
| 153 | 1717 | 5′-TCATCACAAAGAAGTC-3′ | antisense sequence |
| 154 | 1718 | 5′-ATCACAAAGAAGT-3′ | antisense sequence |
| 155 | 1718 | 5′-CATCACAAAGAAGT-3′ | antisense sequence |
| 156 | 1718 | 5′-TCATCACAAAGAAGT-3′ | antisense sequence |
| 157 | 1718 | 5′-TTCATCACAAAGAAGT-3′ | antisense sequence |
| 158 | 1719 | 5′-TTCATCACAAAGAAG-3′ | antisense sequence |
| 159 | 1777 | 5′-ACATCTTCTGAATT-3′ | antisense sequence |
| 160 | 1823 | 5′-GGTGATTGTGACAC-3′ | antisense sequence |
| 161 | 1823 | 5′-GGGTGATTGTGACAC-3′ | antisense sequence |
| 162 | 1823 | 5′-TGGGTGATTGTGACAC-3′ | antisense sequence |
| 163 | 1824 | 5′-GGTGATTGTGACA-3′ | antisense sequence |
| 164 | 1824 | 5′-GGGTGATTGTGACA-3′ | antisense sequence |
| 165 | 1824 | 5′-TGGGTGATTGTGACA-3′ | antisense sequence |
| 166 | 1824 | 5′-GTGGGTGATTGTGACA-3′ | antisense sequence |
| 167 | 1825 | 5′-GGGTGATTGTGAC-3′ | antisense sequence |
| 168 | 1825 | 5′-TGGGTGATTGTGAC-3′ | antisense sequence |
| 169 | 1825 | 5′- [symbol] ^{o}GₛGₛTₛGₛAₛTₛTₛGₛTₛ [symbol] | LNA antisense |
| | | [symbol] -3′ | oligonucleotide |
| 170 | 1825 | 5′-TGTGGGTGATTGTGAC-3′ | antisense sequence |
| 171 | 1825 | 5′-GGTGATTGTGAC-3′ | antisense sequence |
| 172 | 1826 | 5′-TGGGTGATTGTGA-3′ | antisense sequence |
| 173 | 1826 | 5′-GTGGGTGATTGTGA-3′ | antisense sequence |
| 174 | 1826 | 5′-TGTGGGTGATTGTGA-3′ | antisense sequence |
| 175 | 1826 | 5′-GTGTGGGTGATTGTGA-3′ | antisense sequence |
| 176 | 1827 | 5′- [symbol] TₛGₛGₛGₛTₛGₛAₛTₛTₛ | LNA antisense |
| | | [symbol] -3′ | oligonucleotide |
| 177 | 1874 | 5′-GGGACAGTTGTGC-3′ | antisense sequence |
| 178 | 1897 | 5′-TAGAAGTTGAGTTC-3′ | antisense sequence |
| 179 | 1897 | 5′-GTAGAAGTTGAGTTC-3′ | antisense sequence |
| 180 | 1897 | 5′-TGTAGAAGTTGAGTTC-3′ | antisense sequence |
| 181 | 1898 | 5′-GTAGAAGTTGAGTT-3′ | |
| 182 | 1898 | 5′- [symbol] AₛGₛAₛAₛGₛTₛTₛGₛAₛ [symbol] | LNA antisense |
| | | [symbol] -3′ | oligonucleotide |
| 183 | 1898 | 5′-CTGTAGAAGTTGAGTT-3′ | antisense sequence |
| 184 | 1899 | 5′-CTGTAGAAGTTGAGT-3′ | antisense sequence |
| 185 | 1899 | 5′-GCTGTAGAAGTTGAGT-3′ | antisense sequence |
| 186 | 1900 | 5′-CTGTAGAAGTTGAG-3′ | antisense sequence |
| 187 | 1900 | 5′-GCTGTAGAAGTTGAG-3′ | antisense sequence |
| 188 | 1901 | 5′-GCTGTAGAAGTTGA-3′ | antisense sequence |
| 189 | 1946 | 5′-CAAGCTATGATGG-3′ | antisense sequence |
| 190 | 1946 | 5′-GCAAGCTATGATGG-3′ | antisense sequence |
| 191 | 1946 | 5′-TGCAAGCTATGATGG-3′ | antisense sequence |
| 192 | 1946 | 5′-CTGCAAGCTATGATGG-3′ | antisense sequence |
| 193 | 1947 | 5′-TGCAAGCTATGATG-3′ | antisense sequence |
| 194 | 1947 | 5′-CTGCAAGCTATGATG-3′ | antisense sequence |
| 195 | 1948 | 5′-TGCAAGCTATGAT-3′ | antisense sequence |
| 196 | 1948 | 5′-CTGCAAGCTATGAT-3′ | antisense sequence |
| 197 | 2021 | 5′-CTCCTGGCTGATCA-3′ | antisense sequence |
| 198 | 2057 | 5′-AAGGTAGCACAGGC-3′ | antisense sequence |
| 199 | 2057 | 5′-GAAGGTAGCACAGGC-3′ | antisense sequence |
| 200 | 2057 | 5′-AGAAGGTAGCACAGGC-3′ | antisense sequence |
| 201 | 2058 | 5′-GAAGGTAGCACAGG-3′ | antisense sequence |
| 202 | 2058 | 5′-AGAAGGTAGCACAGG-3′ | antisense sequence |
| 203 | 2058 | 5′-GAGAAGGTAGCACAGG-3′ | antisense sequence |
| 204 | 2059 | 5′-AGAAGGTAGCACAG-3′ | antisense sequence |
| 205 | 2059 | 5′-GAGAAGGTAGCACAG-3′ | antisense sequence |
| 206 | 2059 | 5′-AGAGAAGGTAGCACAG-3′ | antisense sequence |
| 207 | 2060 | 5′-AGAAGGTAGCACA-3′ | antisense sequence |
| 208 | 2060 | 5′-GAGAAGGTAGCACA-3′ | antisense sequence |
| 209 | 2060 | 5′-AGAGAAGGTAGCACA-3′ | antisense sequence |
| 210 | 2060 | 5′-GAGAGAAGGTAGCACA-3′ | antisense sequence |
| 211 | 2061 | 5′-AGAAGGTAGCAC-3′ | antisense sequence |
| 212 | 2061 | 5′-GAGAAGGTAGCAC-3′ | antisense sequence |
| 213 | 2061 | 5′-AGAGAAGGTAGCAC-3′ | antisense sequence |
| 214 | 2061 | 5′- [symbol] ^{o}AₛGₛAₛAₛGₛGₛTₛAₛGₛ | LNA antisense |
| | | [symbol] -3′ | oligonucleotide |
| 215 | 2062 | 5′-GAGAGAAGGTAGCA-3′ | antisense sequence |
| 216 | 2063 | 5′-GAGAGAAGGTAGC-3′ | antisense sequence |
| 217 | 2148 | 5′-GAATGCCATACTGG-3′ | antisense sequence |
| 218 | 2148 | 5′-AGAATGCCATACTGG-3′ | antisense sequence |
| 219 | 2149 | 5′-AGAATGCCATACTG-3′ | antisense sequence |
| 220 | 2179 | 5′-ATCTTCCTGGTCATC-3′ | antisense sequence |
| 221 | 2215 | 5′-TTGTCCCACTGCTG-3′ | antisense sequence |
| 222 | 2215 | 5′-CTTGTCCCACTGCTG-3′ | antisense sequence |
| 223 | 2215 | 5′-TCTTGTCCCACTGCTG-3′ | antisense sequence |
| 224 | 2216 | 5′-CTTGTCCCACTGCT-3′ | antisense sequence |
| 225 | 2216 | 5′-TCTTGTCCCACTGCT-3′ | antisense sequence |
| 226 | 2216 | 5′-TTCTTGTCCCACTGCT-3′ | antisense sequence |
| 227 | 2218 | 5′-GCTTCTTGTCCCACTG-3′ | antisense sequence |
| 228 | 2219 | 5′-GCTTCTTGTCCCACT-3′ | antisense sequence |
| 229 | 2219 | 5′-AGCTTCTTGTCCCACT-3′ | antisense sequence |
| 230 | 2220 | 5′-GCTTCTTGTCCCAC-3′ | antisense sequence |
| 231 | 2220 | 5′-AGCTTCTTGTCCCAC-3′ | antisense sequence |
| 232 | 2220 | 5′-AAGCTTCTTGTCCCAC-3′ | antisense sequence |
| 233 | 2221 | 5′-AGCTTCTTGTCCCA-3′ | antisense sequence |
| 234 | 2221 | 5′-AAGCTTCTTGTCCCA-3′ | antisense sequence |
| 235 | 2222 | 5′-AAGCTTCTTGTCCC-3′ | antisense sequence |
| 236 | 2223 | 5′-AAGCTTCTTGTCC-3′ | antisense sequence |
| 237 | 2266 | 5′-ACTGTCTTCATCTTC-3′ | antisense sequence |
| 238 | 2266 | 5′-CACTGTCTTCATCTTC-3′ | antisense sequence |
| 239 | 2269 | 5′-AGTCACTGTCTTCATC-3′ | antisense sequence |
| 240 | 2270 | 5′-AGTCACTGTCTTCAT-3′ | antisense sequence |
| 241 | 2270 | 5′-AAGTCACTGTCTTCAT-3′ | antisense sequence |
| 242 | 2272 | 5′-CAAAGTCACTGTCTTC-3′ | antisense sequence |
| 243 | 2273 | 5′-CAAAGTCACTGTCTT-3′ | antisense sequence |
| 244 | 2273 | 5′-CCAAAGTCACTGTCTT-3′ | antisense sequence |
| 245 | 2274 | 5′-CCAAAGTCACTGTCT-3′ | antisense sequence |
| 246 | 2275 | 5′-CCAAAGTCACTGTC-3′ | antisense sequence |
| 247 | 2298 | 5′-TAGCAATCTCGC-3′ | antisense sequence |
| 248 | 2393 | 5′-AGATGGCAGCAGAGC-3′ | antisense sequence |
| 249 | 2393 | 5′- [symbol] AₛTₛGₛGₛCₛAₛGₛCₛAₛGₛ | LNA antisense |
| | | [symbol] -3′ | oligonucleotide |
| 250 | 2394 | 5'- [symbol] GₛAₛTₛGₛGₛCₛAₛGₛCₛAₛ [symbol] | LNA antisense |
| | | [symbol] -3′ | oligonucleotide |
| 251 | 2395 | 5'-AAAGATGGCAGCAGA-3' | antisense sequence |
| 252 | 2395 | 5'-CAAAGATGGCAGCAGA-3' | antisense sequence |
| 253 | 2396 | 5'-CAAAGATGGCAGCAG-3' | antisense sequence |
| 254 | 2396 | 5'- [symbol] AₛAₛGₛAₛTₛGₛGₛCₛAₛGₛ | LNA antisense |
| | | [symbol] -3' | oligonucleotide |
| 255 | 2656 | 5′-AAACTCAGAATATA-3′ | antisense sequence |
| 256 | 2657 | 5′-AAACTCAGAATAT-3′ | antisense sequence |
| 257 | 2668 | 5′-TACAGCACCACAAA-3′ | antisense sequence |
| 258 | 2668 | 5′-CTACAGCACCACAAA-3′ | antisense sequence |
| 259 | 2669 | 5′-CTACAGCACCACAA-3′ | antisense sequence |
| 260 | 2670 | 5′-CTACAGCACCACA-3′ | antisense sequence |
| 261 | 3006 | 5′-GTCCATCACAGTAA-3′ | antisense sequence |
| 262 | 3006 | 5′-TGTCCATCACAGTAA-3′ | antisense sequence |

“s” represents phosphorothioate linkage, bold letters represents LNA molecules.

Preferred designs of oligonucleotides are 3-10-3, 3-9-3, 3-8-3, 2-8-3, 3-8-2, 2-8-2 of the LNA-DNA-LNA type of gapmers.

A selection of specially preferred antisense sequence motifs.
Oligonucleotides of the invention will preferably comprise
or consist of part of the sequence of any one of the
listed motifs.

| | Start point | | |
|---|---|---|---|
| | of target | | |
| | sequence in | | |
| Test | human mRNA | | Type of |
| substance | as of | | oligonucleotide |
| Seq. ID# | SEQ ID 263 | Sequence motif | sequence |
| 264 | 232 | 5′-GGCAGATAAGAAAC-3′ | Antisense sequence |
| 265 | 452 | 5′-GTTTCATTGATATAAATAACATTCCG | antisense sequence |
| | | CAAACCCA-3′ | |
| 266 | 556 | 5′-CAAACATGCCCTTATG-3′ | antisense sequence |
| 267 | 613 | 5′-CAATTGCCTCTTG-3′ | antisense sequence |
| 268 | 784 | 5′-AAGAAGCTGTTGAA-3′ | antisense sequence |
| 269 | 842 | 5′-TTCGTCTCAGTTGCAG-3′ | antisense sequence |
| 270 | 961 | 5′-GGGATGTTGAGATTATTGCC-3′ | antisense sequence |
| 271 | 1030 | 5′-GTTTCATCGAGCCT-3′ | antisense sequence |
| 272 | 1273 | 5′-ATTGTAGTGACCTTCGAT-3′ | antisense sequence |
| 273 | 1414 | 5′-TTCTAAATATTCCTT-3′ | antisense sequence |
| 274 | 1667 | 5′-ATTCCCTGCCTGTGTCTGTAGAGGA | antisense sequence |
| | | GCA-3′ | |
| 275 | 1716 | 5′-TTCATCACAAAGAAGTCT-3′ | antisense sequence |
| 276 | 1777 | 5′-ACATCTTCTGAATT-3′ | antisense sequence |
| 277 | 1823 | 5′-GTGTGGGTGATTGTGACAC-3′ | antisense sequence |
| 278 | 1874 | 5′-GGGACAGTTGTGC-3′ | antisense sequence |
| 279 | 1897 | 5′-GCTGTAGAAGTTGAGTTC-3′ | antisense sequence |
| 280 | 1946 | 5′-CTGCAAGCTATGATGG-3′ | antisense sequence |
| 281 | 2021 | 5′-CTCCTGGCTGATCA-3′ | antisense sequence |
| 282 | 2057 | 5′-GAGAGAAGGTAGCACAGGC-3′ | antisense sequence |
| 283 | 2148 | 5′-AGAATGCCATACTGG-3′ | antisense sequence |
| 284 | 2179 | 5′-ATCTTCCTGGTCATC-3′ | antisense sequence |
| 285 | 2215 | 5′-AAGCTTCTTGTCCCACTGCTG-3′ | antisense sequence |
| 286 | 2266 | 5′-CCAAAGTCACTGTCTTCATCTTC-3′ | antisense sequence |
| 287 | 2298 | 5′-TAGCAATCTCGC-3′ | antisense sequence |
| 288 | 2393 | 5′-ACAAAGATGGCAGCAGAGC-3′ | antisense sequence |
| 289 | 2656 | 5′-CTACAGCACCACAAAACTCAGAATA | antisense sequence |
| | | TA-3′ | |
| 290 | 3006 | 5′-TGTCCATCACAGTAA-3′ | antisense sequence |

TABLE 3
A selection of specially preferred antisense oligonucleotide
sequences, with sequence ID numbers identical to numbers in
Table 1. Also in this table, the preferred designs of
oligonucleotides are 3-10-3, 3-9-3, 3-8-3, 2-8-3, 3-8-2,
2-8-2 of the LNA-DNA-LNA type of gapmers.

| | Start point of | | |
|---|---|---|---|
| Test | target sequence in | | Type of |
| substance | human mRNA as | | oligonucleotide |
| Sequence ID # | of SEQ ID 263 | Oligonucleotide sequence | sequence |
| 1 | 232 | 5′-GCAGATAAGAAAC-3′ | antisense sequence |
| 2 | 232 | 5′-GGCAGATAAGAAAC-3′ | antisense sequence |
| 28 | 458 | 5′-TAACATTCCGCA-3′ | antisense sequence |
| 29 | 458 | 5′-ATAACATTCCGCA-3′ | antisense sequence |
| 33 | 459 | 5′-ATAACATTCCGC-3′ | antisense sequence |
| 34 | 459 | 5′-AATAACATTCCGC-3′ | antisense sequence |
| 35 | 459 | 5′-AAATAACATTCCGC-3′ | antisense sequence |
| 36 | 459 | 5′-TAAATAACATTCCGC-3′ | antisense sequence |
| 39 | 460 | 5′-AAATAACATTCCG-3′ | antisense sequence |
| 40 | 460 | 5′-TAAATAACATTCCG-3′ | antisense sequence |
| 41 | 460 | 5′-ATAAATAACATTCCG-3′ | antisense sequence |
| 55 | 466 | 5′-TGATATAAATAAC-3′ | antisense sequence |
| 56 | 466 | 5′-TTGATATAAATAAC-3′ | antisense sequence |
| 65 | 556 | 5′-ACATGCCCTTATG-3′ | antisense sequence |
| 80 | 843 | 5′-CGTCTCAGTTGCA-3′ | antisense sequence |
| 86 | 845 | 5′-TTCGTCTCAGTTG-3′ | antisense sequence |
| 87 | 846 | 5′-TTCGTCTCAGTT-3′ | antisense sequence |
| 88 | 961 | 5′-TGAGATTATTGCC-3′ | antisense sequence |
| 92 | 962 | 5′-GTTGAGATTATTGC-3′ | antisense sequence |
| 121 | 1274 | 5′-TGTAGTGACCTTCGA-3′ | antisense sequence |
| 125 | 1275 | 5′-TGTAGTGACCTTCG-3′ | antisense sequence |
| 130 | 1277 | 5′-ATTGTAGTGACCTT-3′ | antisense sequence |
| 131 | 1414 | 5′-TTCTAAATATTCCTT-3′ | antisense sequence |
| 135 | 1674 | 5′-TGCCTGTGTCTGTAG-3′ | antisense sequence |
| 142 | 1678 | 5′-TTCCCTGCCTGTGTCT-3′ | antisense sequence |
| 145 | 1680 | 5′-TTCCCTGCCTGTGT-3′ | antisense sequence |
| 147 | 1681 | 5′-ATTCCCTGCCTGTG-3′ | antisense sequence |
| 151 | 1717 | 5′-ATCACAAAGAAGTC-3′ | antisense sequence |
| 155 | 1718 | 5′-CATCACAAAGAAGT-3′ | antisense sequence |
| 159 | 1777 | 5′-ACATCTTCTGAATT-3′ | antisense sequence |
| 168 | 1825 | 5′-TGGGTGATTGTGAC-3′ | antisense sequence |
| 169 | 1825 | 5′-GTGGGTGATTGTGAC-3′ | antisense sequence |
| 176 | 1827 | 5′-GTGTGGGTGATTGTG-3′ | antisense sequence |
| 181 | 1898 | 5′-GTAGAAGTTGAGTT-3′ | antisense sequence |
| 182 | 1898 | 5′-TGTAGAAGTTGAGTT-3′ | antisense sequence |
| 213 | 2061 | 5′-AGAGAAGGTAGCAC-3′ | antisense sequence |
| 214 | 2061 | 5′-GAGAGAAGGTAGCAC-3′ | antisense sequence |
| 215 | 2062 | 5′-GAGAGAAGGTAGCA-3′ | antisense sequence |
| 216 | 2063 | 5′-GAGAGAAGGTAGC-3′ | antisense sequence |
| 221 | 2215 | 5′-TTGTCCCACTGCTG-3′ | antisense sequence |
| 224 | 2216 | 5′-CTTGTCCCACTGCT-3′ | antisense sequence |
| 249 | 2393 | 5′-AAGATGGCAGCAGAGC-3′ | antisense sequence |
| 250 | 2394 | 5′-AAAGATGGCAGCAGAG-3′ | antisense sequence |
| 254 | 2396 | 5′-ACAAAGATGGCAGCAG-3′ | antisense sequence |
| 257 | 2668 | 5′-TACAGCACCACAAA-3′ | antisense sequence |
| 260 | 2670 | 5′-CTACAGCACCACA-3′ | antisense sequence |
| 261 | 3006 | 5′-GTCCATCACAGTAA-3′ | antisense sequence |

The oligomer may comprise or consist of a contiguous nucleotide sequence which is fully complementary (perfectly complementary) to the equivalent region of a nucleic acid which encodes a mammalian mtGPAT1 (e.g., SEQ ID NO: 263). Thus, the oligomer can comprise or consist of an antisense nucleotide sequence.

However, in some embodiments, the oligomer may tolerate 1, 2, 3, or 4 (or more) mismatches, when hybridising to the target sequence and still sufficiently bind to the target to show the desired effect, i.e. down-regulation of the target. Mismatches may, for example, be compensated by increased length of the oligomer nucleotide sequence and/or an increased number of nucleotide analogues, such as LNA, present within the nucleotide sequence.

In some embodiments, the contiguous nucleotide sequence comprises no more than 3, such as no more than 2 mismatches when hybridizing to the target sequence, such as to the corresponding region of a nucleic acid which encodes a mammalian mtGPAT1.

In some embodiments, the contiguous nucleotide sequence comprises no more than a single mismatch when hybridizing to the target sequence, such as the corresponding region of a nucleic acid which encodes a mammalian mtGPAT1.

The nucleotide sequence of the oligomers of the invention or the contiguous nucleotide sequence is preferably at least 80% homologous to the of a corresponding sequence selected from the group consisting of SEQ ID NOS: 1-262, such as at least 85%, at least 90%, at least 91%, at least 92%, at least 93%, at least 94%, at least 95%, at least 96% homologous, such as 100% homologous (identical).

The nucleotide sequence of the oligomers of the invention or the contiguous nucleotide sequence is preferably at least 80% homologous to the reverse complement of a corresponding sequence present in SEQ ID NO: 263, such as at least 85%, at least 90%, at least 91%, at least 92% at least 93%, at least 94%, at least 95%, at least 96% homologous, such as 100% homologous (identical).

The nucleotide sequence of the oligomers of the invention or the contiguous nucleotide sequence is preferably at least 80% complementary to a sub-sequence present in SEQ ID NO: 263, such as at least 85%, at least 90%, at least 91%, at least 92%, at least 93%, at least 94%, at least 95%, at least 96% complementary, such as 100% complementary (perfectly complementary).

In some embodiments the oligomer (or contiguous nucleotide portion thereof) is selected from, or comprises, one of the sequences selected from the group consisting of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, or a sub-sequence of at least 10 contiguous nucleotides thereof, wherein said oligomer (or contiguous nucleotide portion thereof) may optionally comprise one, two, or three mismatches when compared to the sequence.

In some embodiments the sub-sequence may consist of 11, 12, 13, 14, 15, 16, 17, 18, 19, 20, 21, 22, 23, 24, 25, 26, 27, 28, or 29 contiguous nucleotides, such as between 12-22, such as between 12-18 nucleotides. Suitably, in some embodiments, the sub-sequence is of the same length as the contiguous nucleotide sequence of the oligomer of the invention.

However, it is recognised that, in some embodiments the nucleotide sequence of the oligomer may comprise additional 5′ or 3′ nucleotides, such as, independently, 1, 2, 3, 4 or 5 additional nucleotides 5′ and/or 3′, which are non-complementary to the target sequence. In this respect the oligomer of the invention, may, in some embodiments, comprise a contiguous nucleotide sequence which is flanked 5′ and or 3′ by additional nucleotides. In some embodiments the additional 5′ or 3′ nucleotides are naturally occurring nucleotides, such as DNA or RNA. In some embodiments, the additional 5′ or 3′ nucleotides may represent region D as referred to in the context of gapmer oligomers herein.

In some embodiments the oligomer according to the invention consists or comprises of a nucleotide sequence according to SEQ ID NO:264, or a sub-sequence of thereof.

In some embodiments the oligomer according to the invention consists or comprises of a nucleotide sequence according to SEQ ID NO:265, or a sub-sequence of thereof.

In some embodiments the oligomer according to the invention consists or comprises of a nucleotide sequence according to SEQ ID NO:266, or a sub-sequence of thereof.

In some embodiments the oligomer according to the invention consists or comprises of a nucleotide sequence according to SEQ ID NO:267, or a sub-sequence of thereof.

In some embodiments the oligomer according to the invention consists or comprises of a nucleotide sequence according to SEQ ID NO: 268, or a sub-sequence of thereof.

In some embodiments the oligomer according to the invention consists or comprises of a nucleotide sequence according to SEQ ID NO: 269, or a sub-sequence of thereof.

In some embodiments the oligomer according to the invention consists or comprises of a nucleotide sequence according to SEQ ID NO: 270, or a sub-sequence of thereof.

In some embodiments the oligomer according to the invention consists or comprises of a nucleotide sequence according to SEQ ID NO: 271, or a sub-sequence of thereof.

In some embodiments the oligomer according to the invention consists or comprises of a nucleotide sequence according to SEQ ID NO: 272, or a sub-sequence of thereof.

In some embodiments the oligomer according to the invention consists or comprises of a nucleotide sequence according to SEQ ID NO: 273, or a sub-sequence of thereof.

In some embodiments the oligomer according to the invention consists or comprises of a nucleotide sequence according to SEQ ID NO: 274, or a sub-sequence of thereof.

In some embodiments the oligomer according to the invention consists or comprises of a nucleotide sequence according to SEQ ID NO: 275, or a sub-sequence of thereof.

In some embodiments the oligomer according to the invention consists or comprises of a nucleotide sequence according to SEQ ID NO: 276, or a sub-sequence of thereof.

In some embodiments the oligomer according to the invention consists or comprises of a nucleotide sequence according to SEQ ID NO: 277, or a sub-sequence of thereof.

In some embodiments the oligomer according to the invention consists or comprises of a nucleotide sequence according to SEQ ID NO: 278, or a sub-sequence of thereof.

In some embodiments the oligomer according to the invention consists or comprises of a nucleotide sequence according to SEQ ID NO: 279, or a sub-sequence of thereof.

In some embodiments the oligomer according to the invention consists or comprises of a nucleotide sequence according to SEQ ID NO: 280, or a sub-sequence of thereof.

In some embodiments the oligomer according to the invention consists or comprises of a nucleotide sequence according to SEQ ID NO: 281, or a sub-sequence of thereof.

In some embodiments the oligomer according to the invention consists or comprises of a nucleotide sequence according to SEQ ID NO: 282, or a sub-sequence of thereof.

In some embodiments the oligomer according to the invention consists or comprises of a nucleotide sequence according to SEQ ID NO: 283, or a sub-sequence of thereof.

In some embodiments the oligomer according to the invention consists or comprises of a nucleotide sequence according to SEQ ID NO: 284, or a sub-sequence of thereof.

In some embodiments the oligomer according to the invention consists or comprises of a nucleotide sequence according to SEQ ID NO: 285, or a sub-sequence of thereof.

In some embodiments the oligomer according to the invention consists or comprises of a nucleotide sequence according to SEQ ID NO: 286, or a sub-sequence of thereof.

In some embodiments the oligomer according to the invention consists or comprises of a nucleotide sequence according to SEQ ID NO: 287, or a sub-sequence of thereof.

In some embodiments the oligomer according to the invention consists or comprises of a nucleotide sequence according to SEQ ID NO: 288, or a sub-sequence of thereof.

In some embodiments the oligomer according to the invention consists or comprises of a nucleotide sequence according to SEQ ID NO: 289, or a sub-sequence of thereof.

In some embodiments the oligomer according to the invention consists or comprises of a nucleotide sequence according to SEQ ID NO: 290, or a sub-sequence of thereof.

In one preferred embodiment, the oligomer of the invention is any one of SEQ ID NO's 2, 33, 125, 142, 147, 169, 176, 182, 214, 249, 250 and 254.

When determining “homology” between the oligomers of the invention (or contiguous nucleotide sequence) and the nucleic acid which encodes the mammalian mtGPAT1 or the reverse complement thereof, such as those disclosed herein, the determination of homology may be made by a simple alignment with the corresponding nucleotide sequence of the compound of the invention and the corresponding region of the nucleic acid which encodes the mammalian mtGPAT1 (or target nucleic acid), or the reverse complement thereof, and the homology is determined by counting the number of bases which align and dividing by the total number of contiguous nucleotides in the compound of the invention, and multiplying by 100. In such a comparison, if gaps exist, it is preferable that such gaps are merely mismatches rather than areas where the number of nucleotides within the gap differ between the nucleotide sequence of the invention and the target nucleic acid.

The terms “corresponding to” and “corresponds to” refer to the comparison between the nucleotide sequence of the oligomer or contiguous nucleotide sequence (a first sequence) and the equivalent contiguous nucleotide sequence of a further sequence selected from either i) a sub-sequence of the reverse complement of the nucleic acid target, such as the mRNA which encodes the mtGPAT1 protein, such as SEQ ID NO: 263, and/or ii) the sequence of nucleotides provided herein such as the group consisting of SEQ ID NOS: 264, 265, 266, 267, 268, 269, 270, 271, 272, 273, 274, 275, 276, 277, 278, 279, 280, 281, 282, 283, 284, 285, 286, 287, 288, 289, and 290. Nucleotide analogues are compared directly to their equivalent or corresponding nucleotides. A first sequence which corresponds to a further sequence under i) or ii) typically is identical to that sequence over the length of the first sequence (such as the contiguous nucleotide sequence) or, as described herein may, in some embodiments, is at least 80% homologous to a corresponding sequence, such as at least 85%, at least 90%, at least 91%, at least 92% at least 93%, at least 94%, at least 95%, at least 96% homologous, such as 100% homologous (identical).

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

### Length

The oligomers comprise or consist of a contiguous nucleotide sequence of a total of between 10, 11, 12, 13, 14, 15, 16, 17, 18, 19, 20, 21, 22, 23, 24, 25, 26, 27, 28, 29 or 30 contiguous nucleotides in length.

In some embodiments, the oligomers comprise or consist of a contiguous nucleotide sequence of a total of between 10-22, such as 12-18, such as 13-17 or 12-16, such as 13, 14, 15, 16 contiguous nucleotides in length.

In some embodiments, the oligomers comprise or consist of a contiguous nucleotide sequence of a total of 10, 11, 12, 13, or 14 contiguous nucleotides in length.

In some embodiments, the oligomer according to the invention consists of no more than 22 nucleotides, such as no more than 20 nucleotides, such as no more than 18 nucleotides, such as 15, 16 or 17 nucleotides. In some embodiments the oligomer of the invention comprises less than 20 nucleotides.

### Nucleotide Analogues

The term “nucleotide” as used herein, refers to a glycoside comprising a sugar moiety, a base moiety and a covalently linked group, such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogues” herein. Herein, a single nucleotide (unit) may also be referred to as a monomer or nucleic acid unit.

In field of biochemistry, the term “nucleoside” is commonly used to refer to a glycoside comprising a sugar moiety and a base moiety, and may therefore be used when referring to the nucleotide units, which are covalently linked by the internucleotide linkages between the nucleotides of the oligomer.

As one of ordinary skill in the art would recognise, the 5′ nucleotide of an oligonucleotide does not comprise a 5′ internucleotide linkage group, although may or may not comprise a 5′ terminal group.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

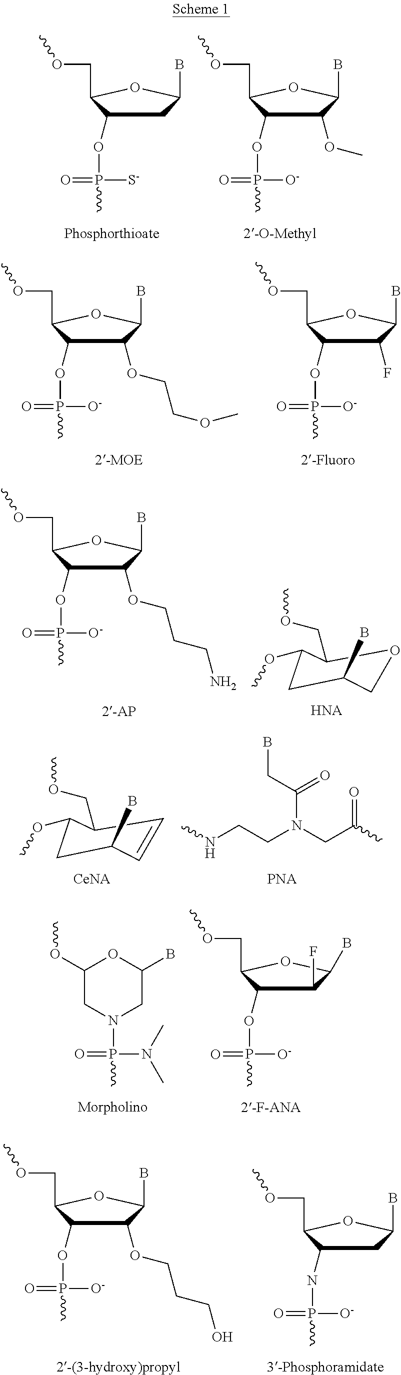

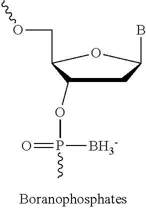

The oligomer may thus comprise or consist of a simple sequence of natural occurring nucleotides—preferably 2′-deoxynucleotides (referred to here generally as “DNA”), but also possibly ribonucleotides (referred to here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

Examples of suitable and preferred nucleotide analogues are provided by PCT/DK2006/000512 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

In some embodiments the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

It will be recognised that when referring to a preferred nucleotide sequence motif or nucleotide sequence, which consists of only nucleotides, the oligomers of the invention which are defined by that sequence may comprise a corresponding nucleotide analogue in place of one or more of the nucleotides present in said sequence, such as LNA units or other nucleotide analogues, which raise the duplex stability/Tₘ of the oligomer/target duplex (i.e. affinity enhancing nucleotide analogues).

In some embodiments, any mismatches between the nucleotide sequence of the oligomer and the target sequence are preferably found in regions outside the affinity enhancing nucleotide analogues, such as region B as referred to herein, and/or region D as referred to herein, and/or at the site of non modified such as DNA nucleotides in the oligonucleotide, and/or in regions which are 5′ or 3′ to the contiguous nucleotide sequence.

Examples of such modification of the nucleotide include modifying the sugar moiety to provide a 2′-substituent group or to produce a bridged (locked nucleic acid) structure which enhances binding affinity and may also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligomer of the invention (such as in regions A and C mentioned herein) are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid—Christensen, 2002. Nucl. Acids. Res. 2002 30: 4918-4925, hereby incorporated by reference) units and 2′MOE units. In some embodiments there is only one of the above types of nucleotide analogues present in the oligomer of the invention, or contiguous nucleotide sequence thereof.

In some embodiments the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In some embodiments at least one of said nucleotide analogues is 2′-MOE-RNA, such as 2, 3, 4, 5, 6, 7, 8, 9 or 10 2′-MOE-RNA nucleotide units. In some embodiments at least one of said nucleotide analogues is 2′-fluoro DNA, such as 2, 3, 4, 5, 6, 7, 8, 9 or 10 2′-fluoro-DNA nucleotide units.

In some embodiments, the oligomer according to the invention comprises at least one Locked Nucleic Acid (LNA) unit, such as 1, 2, 3, 4, 5, 6, 7, or 8 LNA units, such as between 3-7 or 4 to 8 LNA units, or 3, 4, 5, 6 or 7 LNA units. In some embodiments, all the nucleotide analogues are LNA. In some embodiments, the oligomer may comprise both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5′ methyl-Cytosine. In some embodiments of the invention, the oligomer may comprise both LNA and DNA units. Preferably the combined total of LNA and DNA units is 10-25, preferably 10-20, even more preferably 12-16. In some embodiments of the invention, the nucleotide sequence of the oligomer, such as the contiguous nucleotide sequence consists of at least one LNA and the remaining nucleotide units are DNA units. In some embodiments the oligomer comprises only LNA nucleotide analogues and naturally occurring nucleotides (such as RNA or DNA, most preferably DNA nucleotides), optionally with modified internucleotide linkages such as phosphorothioate.

The term “nucleobase” refers to the base moiety of a nucleotide and covers both naturally occurring a well as non-naturally occurring variants. Thus, “nucleobase” covers not only the known purine and pyrimidine heterocycles but also heterocyclic analogues and tautomers thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### LNA

The term “LNA” refers to a bicyclic nucleotide analogue, known as “Locked Nucleic Acid”. It may refer to an LNA monomer, or, when used in the context of an “LNA oligonucleotide”, LNA refers to an oligonucleotide containing one or more such bicyclic nucleotide analogues. LNA nucleotides are characterised by the presence of a biradical ‘bridge’ between C2′ and C4′ of the ribose sugar ring—for example as shown as the biradical R⁴*-R²* as described below.

The LNA used in the oligonucleotide compounds of the invention preferably has the structure of the general formula I

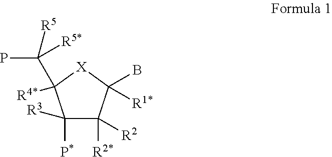

wherein for all chiral centers, asymmetric groups may be found in either R or S orientation;

wherein X is selected from —O—, —S—, —N(R^{N}*)—, —C(R⁶R⁶*)—, such as, in some embodiments —O—;

B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands;

P designates an internucleotide linkage to an adjacent monomer, or a 5′-terminal group, such internucleotide linkage or 5′-terminal group optionally including the substituent R⁵ or equally applicable the substituent R⁵*;

P* designates an internucleotide linkage to an adjacent monomer, or a 3′-terminal group;

R⁴* and R²* together designate a biradical consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z, wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, optionally substituted C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), wherein for all chiral centers, asymmetric groups may be found in either R or S orientation, and;

each of the substituents R¹*, R², R³, R⁵, R⁵*, R⁶ and R⁶*, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene; wherein R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof. For all chiral centers, asymmetric groups may be found in either R or S orientation.

In some embodiments, R⁴* and R²* together designate a biradical consisting of a groups selected from the group consisting of C(R^{a}R^{b})—C(R^{a}R^{b})—, C(R^{a}R^{b})—O—, C(R^{a}R^{b})—NR^{a}—, C(R^{a}R^{b})—S—, and C(R^{a}R^{b})—C(R^{a}R^{b})—O—, wherein each R^{a} and R^{b} may optionally be independently selected. In some embodiments, R^{a} and R^{b} may be, optionally independently selected from the group consisting of hydrogen and C₁₋₆alkyl, such as methyl, such as hydrogen.

In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆-aminoalkyl or substituted C₁₋₆-aminoalkyl. For all chiral centers, asymmetric groups may be found in either R or S orientation.

In some embodiments, R¹*, R², R³, R⁵, R⁵* are hydrogen.

In some embodiments, R¹*, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups may be found in either R or S orientation.

In some embodiments, R¹*, R², R³ are hydrogen.

In some embodiments, R⁵ and R⁵* are each independently selected from the group consisting of H, —CH₃, —CH₂—CH₃, —CH₂—O—CH₃, and —CH═CH₂. Suitably in some embodiments, either R⁵ or R⁵* are hydrogen, where as the other group (R⁵ or R⁵* respectively) is selected from the group consisting of C₁₋₅ alkyl, C₂₋₆ alkenyl, C₂₋₆ alkynyl, substituted C₁₋₆ alkyl, substituted C₂₋₆ alkenyl, substituted C₂₋₆ alkynyl or substituted acyl (—C(═O)—); wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, OJ₁, SJ₁, NJ₁J₂, N₃, COOJ₁, CN, O—C(═O)NJ₁J₂, N(H)C(═NH)NJ, J₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, C₁₋₆ aminoalkyl, substituted C₁₋₆ aminoalkyl or a protecting group. In some embodiments either R⁵ or R⁵* is substituted C₁₋₆ alkyl. In some embodiments either R⁵ or R⁵* is substituted methylene wherein preferred substituent groups include one or more groups independently selected from F, NJ₁J₂, N₃, CN, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R⁵* is methyl, ethyl or methoxymethyl. In some embodiments either R⁵ or R⁵* is methyl. In a further embodiment either R⁵ or R⁵* is ethylenyl. In some embodiments either R⁵ or R⁵* is substituted acyl. In some embodiments either R⁵ or R⁵* is C(═O)NJ₁J₂. For all chiral centers, asymmetric groups may be found in either R or S orientation. Such 5′ modified bicyclic nucleotides are disclosed in WO 2007/134181, which is hereby incorporated by reference in its entirety.

In some embodiments B is a nucleobase, including nucleobase analogues and naturally occurring nucleobases, such as a purine or pyrimidine, or a substituted purine or substituted pyrimidine, such as a nucleobase referred to herein, such as a nucleobase selected from the group consisting of adenine, cytosine, thymine, adenine, uracil, and/or a modified or substituted nucleobase, such as 5-thiazolo-uracil, 2-thio-uracil, 5-propynyl-uracil, 2′ thio-thymine, 5-methyl cytosine, 5-thiazolo-cytosine, 5-propynyl-cytosine, and 2,6-diaminopurine.

In some embodiments, R⁴* and R²* together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R^{d})—S—, wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂). For all chiral centers, asymmetric groups may be found in either R or S orientation.

In a further embodiment R⁴* and R²* together designate a biradical (bivalent group) selected from —CH₂—O—, —CH₂—S—, —CH₂—NH—, —CH₂—N(CH₃)—, —CH₂—OH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, and —CH(CH₂—O—CH₃)—O—, and/or, —CH₂—CH₂—, and —CH═CH—For all chiral centers, asymmetric groups may be found in either R or S orientation.

In some embodiments, R⁴* and R²* together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen, and; wherein R^{c} is selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* together designate the biradical C(R^{a}R^{b})—O—C(R^{c}R^{d})—O—, wherein R^{a}, R^{b}, R^{c}, and R^{d} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl. In some embodiments Z is substituted C₁₋₆ alkyl. In some embodiments said substituent group is C₁₋₆ alkoxy. In some embodiments Z is CH₃OCH₂—. For all chiral centers, asymmetric groups may be found in either R or S orientation. Such bicyclic nucleotides are disclosed in U.S. Pat. No. 7,399,845 which is hereby incorporated by reference in its entirety. In some embodiments, R¹*, R², R³, R⁵, R⁵* are hydrogen. In some embodiments, R¹*, R², R³* are hydrogen, and one or both of R⁵, R⁵* may be other than hydrogen as referred to above and in WO 2007/134181.

In some embodiments, R⁴* and R²* together designate a biradical which comprise a substituted amino group in the bridge such as consist or comprise of the biradical —CH₂—N(R^{c})—, wherein R^{c} is C₁₋₁₂ alkyloxy. In some embodiments R⁴* and R²* together designate a biradical —Cq₃q₄-NOR—, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, O—C(═O)NJ₁J₂, N(H)C(═NH)NJ₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups may be found in either R or S orientation. Such bicyclic nucleotides are disclosed in WO2008/150729 which is hereby incorporated by reference in its entirety. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* may be other than hydrogen as referred to above and in WO 2007/134181. In some embodiments R⁴* and R²* together designate a biradical (bivalent group) C(R^{a}R^{b})—O—, wherein R^{a} and R^{b} are each independently halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁SJ₁, SOJ₁, SO₂J₁, NJ₁J₂, N₃, CN, C(═O)OJ₁, C(═O)NJ₁J₂, C(═O)J₁, O—C(═O)NJ₁J₂, N(H)C(═NH)NJ₁J₂, N(H)C(═O)NJ₁J₂ or N(H)C(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, CN, C(═O)OJ₁, C(═O)NJ₁J₂, C(═O)J₁, O—C(═O)NJ₁J₂, N(H)C(═O)NJ₁J₂ or N(H)C(═S)NJ₁J₂ and; each J₁ and J₂ is, independently, H, C1-C₆ alkyl, substituted C1-C₆ alkyl, C₂-C₆ alkenyl, substituted C₂-C₆ alkenyl, C₂-C₆ alkynyl, substituted C₂-C₆ alkynyl, C1-C₆ aminoalkyl, substituted C1-C₆ aminoalkyl or a protecting group. Such compounds are disclosed in WO2009006478A, hereby incorporated in its entirety by reference.

In some embodiments, R⁴* and R²* form the biradical -Q-, wherein Q is C(q₁)(q₂)C(q₃)(q₄), C(q₁)=C(q₃), C[═C(q₁)(q₂)]-C(q₃)(q₄) or C(q₁)(q₂)-C[═C(q₃)(q₄)]; q₁, q₂, q₃, q₄ are each independently. H, halogen, C₁₋₁₂ alkyl, substituted C₁₋₁₂ alkyl, C₂₋₁₂ alkenyl, substituted C₁₋₁₂ alkoxy, OJ₁, SJ₁, SOJ₁, SO₂J₁, NJ₁J₂, N₃, CN, C(═O)OJ₁, C(═O)—NJ₁J₂, C(═O) J₁, —C(═O)NJ₁J₂, N(H)C(═NH)NJ₁J₂, N(H)C(═O)NJ₁J₂ or N(H)C(═S)NJ₁J₂; each J₁ and J₂ is, independently, H, C₁₋₆ alkyl, C₂₋₆ alkenyl, C₂₋₆ alkynyl, C₁₋₆ aminoalkyl or a protecting group; and, optionally wherein when Q is C(q₁)(q₂)(q₃)(q₄) and one of q₃ or q₄ is CH₃ then at least one of the other of q₃ or q₄ or one of q₁ and q₂ is other than H. In some embodiments, R¹*, R², R³, R⁵, R⁵* are hydrogen. For all chiral centers, asymmetric groups may be found in either R or S orientation. Such bicyclic nucleotides are disclosed in WO2008/154401 which is hereby incorporated by reference in its entirety. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* may be other than hydrogen as referred to above and in WO 2007/134181 or WO2009/067647 (alpha-L-bicyclic nucleic acids analogs).

In some embodiments the LNA used in the oligonucleotide compounds of the invention preferably has the structure of the general formula II:

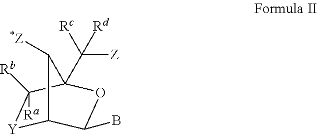

wherein Y is selected from the group consisting of —O—, —CH₂O—, —S—, —NH—, N(R^{e}) and/or —CH₂—; Z and Z* are independently selected among an internucleotide linkage, R^{H}, a terminal group or a protecting group; B constitutes a natural or non-natural nucleotide base moiety (nucleobase), and R^{H} is selected from hydrogen and C₁₋₄-alkyl; R^{a}, R^{b}, R^{c}, R^{d} and R^{e} are, optionally independently, selected from the group consisting of hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂); and R^{H} is selected from hydrogen and C₁₋₄-alkyl. In some embodiments R^{a}, R^{b}R^{c}, R^{d} and R^{e} are, optionally independently, selected from the group consisting of hydrogen and C₁₋₆ alkyl, such as methyl. For all chiral centers, asymmetric groups may be found in either R or S orientation, for example, two exemplary stereochemical isomers include the beta-D and alpha-L isoforms, which may be illustrated as follows:

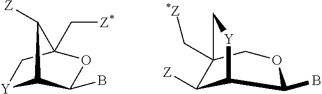

Specific exemplary LNA units are shown below:

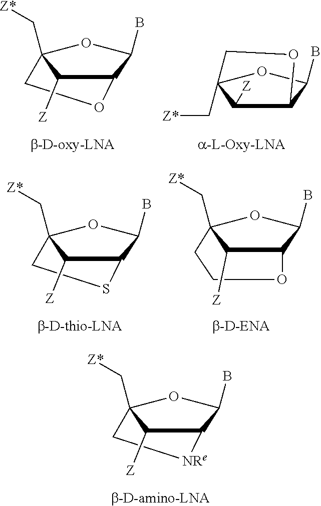

The term “thio-LNA” comprises a locked nucleotide in which Y in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O—. Oxy-LNA can be in both beta-D and alpha-L-configuration.

The term “ENA” comprises a locked nucleotide in which Y in the general formula above is —CH₂—O— (where the oxygen atom of —CH₂—O— is attached to the 2′-position relative to the base B). R^{e} is hydrogen or methyl.

In some exemplary embodiments LNA is selected from beta-D-oxy-LNA, alpha-L-oxy-LNA, beta-D-amino-LNA and beta-D-thio-LNA, in particular beta-D-oxy-LNA.

### RNAse Recruitment

It is recognised that an oligomeric compound may function via non RNase mediated degradation of target mRNA, such as by steric hindrance of translation, or other methods, however, the preferred oligomers of the invention are capable of recruiting an endoribonuclease (RNase), such as RNase H.

It is preferable that the oligomer, or contiguous nucleotide sequence, comprises of a region of at least 6, such as at least 7 consecutive nucleotide units, such as at least 8 or at least 9 consecutive nucleotide units (residues), including 7, 8, 9, 10, 11, 12, 13, 14, 15 or 16 consecutive nucleotides, which, when formed in a duplex with the complementary target RNA is capable of recruiting RNase. The contiguous sequence which is capable of recruiting RNAse may be region B as referred to in the context of a gapmer as described herein. In some embodiments the size of the contiguous sequence which is capable of recruiting RNAse, such as region B, may be higher, such as 10, 11, 12, 13, 14, 15, 16, 17, 18, 19 or 20 nucleotide units.

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothioate linkage groups between all nucleotides in the oligonucleotide, using the methodology provided by Example 91-95 of EP 1 222 309.

In some embodiments, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

In other embodiments, an oligomer is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

Typically the region of the oligomer which forms the consecutive nucleotide units which, when formed in a duplex with the complementary target RNA is capable of recruiting RNase consists of nucleotide units which form a DNA/RNA like duplex with the RNA target—and include both DNA units and LNA units which are in the alpha-L configuration, particularly preferred being alpha-L-oxy LNA.

The oligomer of the invention may comprise a nucleotide sequence which comprises both nucleotides and nucleotide analogues, and may be in the form of a gapmer, a headmer or a mixmer.

A headmer is defined by a contiguous stretch of non-RNase recruiting nucleotide analogues at the 5′-end followed by a contiguous stretch of DNA or modified nucleotide units recognizable and cleavable by the RNase towards the 3′-end (such as at least 7 such nucleotides), and a tailmer is defined by a contiguous stretch of DNA or modified nucleotides recognizable and cleavable by the RNase at the 5′-end (such as at least 7 such nucleotides), followed by a contiguous stretch of non-RNase recruiting nucleotide analogues towards the 3′-end. Other chimeras according to the invention, called mixmers consisting of an alternate composition of DNA or modified nucleotides recognizable and cleavable by RNase and non-RNase recruiting nucleotide analogues. Some nucleotide analogues may also be able to mediate RNaseH binding and cleavage. Since α-L-LNA recruits RNaseH activity to a certain extent, smaller gaps of DNA or modified nucleotides recognizable and cleavable by the RNaseH for the gapmer construct might be required, and more flexibility in the mixmer construction might be introduced.

### Gapmer Design

Preferably, the oligomer of the invention is a gapmer. A gapmer oligomer is an oligomer which comprises a contiguous stretch of nucleotides which is capable of recruiting an RNAse, such as RNAseH, such as a region of at least 6 or 7 DNA nucleotides, referred to herein in as region B, wherein region B is flanked both 5′ and 3′ by regions of affinity enhancing nucleotide analogues, such as between 1-6 nucleotide analogues 5′ and 3′ to the contiguous stretch of nucleotides which is capable of recruiting RNAse—these regions are referred to as regions A and C respectively.

Preferably the gapmer comprises a (poly)nucleotide sequence of formula (5′ to 3′), A-B-C, or optionally A-B-C-D or D-A-B-C, wherein; region A (5′ region) consists or comprises of at least one nucleotide analogue, such as at least one LNA unit, such as between 1-6 nucleotide analogues, such as LNA units, and; region B consists or comprises of at least five consecutive nucleotides which are capable of recruiting RNAse (when formed in a duplex with a complementary RNA molecule, such as the mRNA target), such as DNA nucleotides, and; region C (3′ region) consists or comprises of at least one nucleotide analogue, such as at least one LNA unit, such as between 1-6 nucleotide analogues, such as LNA units, and; region D, when present consists or comprises of 1, 2 or 3 nucleotide units, such as DNA nucleotides.

In some embodiments, region A consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units; and/or region C consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units.

In some embodiments B consists or comprises of 5, 6, 7, 8, 9, 10, 11 or 12 consecutive nucleotides which are capable of recruiting RNAse, or between 6-10, or between 7-9, such as 8 consecutive nucleotides which are capable of recruiting RNAse. In some embodiments region B consists or comprises at least one DNA nucleotide unit, such as 1-12 DNA units, preferably between 4-12 DNA units, more preferably between 6-10 DNA units, such as between 7-10 DNA units, most preferably 8, 9 or 10 DNA units.

In some embodiments region A consist of 3 or 4 nucleotide analogues, such as LNA, region B consists of 7, 8, 9 or 10 DNA units, and region C consists of 3 or 4 nucleotide analogues, such as LNA. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 nucleotide units, such as DNA units.

Further gapmer designs are disclosed in WO2004/046160 and are hereby incorporated by reference.

US provisional application, 60/977,409, hereby incorporated by reference, refers to ‘shortmer’ gapmer oligomers, which, in some embodiments may be the gapmer oligomer according to the present invention.

In some embodiments the oligomer is consisting of a contiguous nucleotide sequence of a total of 10, 11, 12, 13 or 14 nucleotide units, wherein the contiguous nucleotide sequence is of formula (5′-3′), A-B-C, or optionally A-B-C-D or D-A-B-C, wherein; A consists of 1, 2 or 3 nucleotide analogue units, such as LNA units; B consists of 7, 8 or 9 contiguous nucleotide units which are capable of recruiting RNAse when formed in a duplex with a complementary RNA molecule (such as a mRNA target); and C consists of 1, 2 or 3 nucleotide analogue units, such as LNA units. When present, D consists of a single DNA unit.

In some embodiments A consists of 1 LNA unit. In some embodiments A consists of 2 LNA units. In some embodiments A consists of 3 LNA units. In some embodiments C consists of 1 LNA unit. In some embodiments C consists of 2 LNA units. In some embodiments C consists of 3 LNA units. In some embodiments B consists of 7 nucleotide units. In some embodiments B consists of 8 nucleotide units. In some embodiments B consists of 9 nucleotide units. In some embodiments B comprises of between 1-9 DNA units, such as 2, 3, 4, 5, 6, 7 or 8 DNA units. In some embodiments B consists of DNA units. In some embodiments B comprises of at least one LNA unit which is in the alpha-L configuration, such as 2, 3, 4, 5, 6, 7, 8 or 9 LNA units in the alpha-L-configuration. In some embodiments B comprises of at least one alpha-L-oxy LNA unit or wherein all the LNA units in the alpha-L-configuration are alpha-L-oxy LNA units. In some embodiments the number of nucleotides present in A-B-C are selected from the group consisting of (nucleotide analogue units-region B-nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1. In some embodiments the number of nucleotides in A-B-C are selected from the group consisting of: 2-7-1, 1-7-2, 2-7-2, 3-7-3, 2-7-3, 3-7-2, 3-7-4, and 4-7-3. In some embodiments both A and C consists of two LNA units each, and B consists of 8 or 9 nucleotide units, preferably DNA units.

### Internucleotide Linkages

The terms “linkage group” or “internucleotide linkage” are intended to mean a group capable of covalently coupling together two nucleotides, two nucleotide analogues, and a nucleotide and a nucleotide analogue, etc. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within PCT/DK2006/000512, for example the internucleotide linkages listed on the first paragraph of page 34 of PCT/DK2006/000512 (hereby incorporated by reference).

It is, in some embodiments, preferred to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred. Phosphorothioate internucleotide linkages are also preferred, particularly for the gap region (B) of gapmers. Phosphorothioate linkages may also be used for the flanking regions (A and C, and for linking A or C to D, and within region D, as appropriate).

Regions A, B and C, may however comprise internucleotide linkages other than phosphorothioate, such as phosphodiester linkages, particularly, for instance when the use of nucleotide analogues protects the internucleotide linkages within regions A and C from endo-nuclease degradation—such as when regions A and C comprise LNA nucleotides.

The internucleotide linkages in the oligomer may be phosphodiester, phosphorothioate or boranophosphate so as to allow RNase H cleavage of targeted RNA. Phosphorothioate is preferred, for improved nuclease resistance and other reasons, such as ease of manufacture.

In one aspect of the oligomer of the invention, the nucleotides and/or nucleotide analogues are linked to each other by means of phosphorothioate groups.

It is recognised that the inclusion of phosphodiester linkages, such as one or two linkages, into an otherwise phosphorothioate oligomer, particularly between or adjacent to nucleotide analogue units (typically in region A and or C) can modify the bioavailability and/or bio-distribution of an oligomer—see WO2008/053314, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleotide linkage groups are phosphorothioate. When referring to specific gapmer oligonucleotide sequences, such as those provided herein it will be understood that, in various embodiments, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C residues are annotated as 5′ methyl modified cytosine, in various embodiments, one or more of the Cs present in the oligomer may be unmodified C residues. in some embodiments in some embodiments

### Oligomeric Compounds

The sequences of the oligomers of the invention may, for example, be selected from the group consisting of: SEQ IDS: 1-262 and 264-290.

### Conjugates

In the context the term “conjugate” is intended to indicate a heterogenous molecule formed by the covalent attachment (“conjugation”) of the oligomer as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligomer of the invention may comprise both a polynucleotide region which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In various embodiments of the invention the oligomeric compound is linked to ligands/conjugates, which may be used, e.g. to increase the cellular uptake of oligomeric compounds. WO2007/031091 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

Conjugation (to a conjugate moiety) may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example between 1-50, such as 2-20 such as 3-10 amino acid residues in length, and/or polyalkylene oxide such as polyethylglycol (PEG) or polypropylene glycol—see WO 2008/034123, hereby incorporated by reference. Suitably the positively charged polymer, such as a polyalkylene oxide may be attached to the oligomer of the invention via a linker such as the releasable linker described in WO 2008/034123.

By way of example, the following conjugate moieties may be used in the conjugates of the invention:

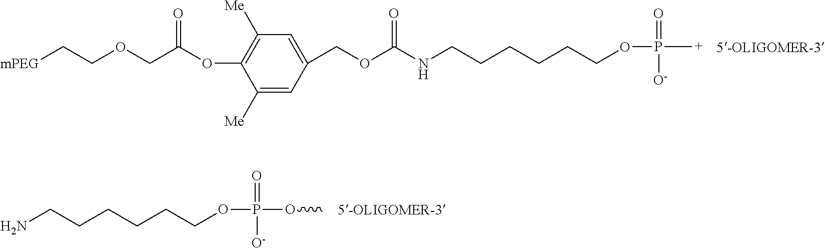

### Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis. In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)_{w}SH)

In some embodiments, sulfhydryl-activated oligonucleotides are conjugated with polymer moieties such as polyethylene glycol or peptides (via formation of a disulfide bond).

Activated oligomers containing hindered esters as described above can be synthesized by any method known in the art, and in particular by methods disclosed in PCT Publication No. WO 2008/034122 and the examples therein, which is incorporated herein by reference in its entirety.

In still other embodiments, the oligomers of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the invention have a functionalizing reagent coupled to a hydroxyl group on the backbone of the oligomer. In yet further embodiments, the oligomer of the invention is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers of the invention may have amine-containing functional moieties on the nucleobase, including on the N6 purine amino groups, on the exocyclic N2 of guanine, or on the N4 or 5 positions of cytosine. In various embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.). Compositions

The oligomer of the invention may be used in pharmaceutical formulations and compositions. Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512—which are also hereby incorporated by reference.

### Applications

The oligomers of the invention may be utilized as research reagents for, for example, diagnostics, therapeutics and prophylaxis.

In research, such oligomers may be used to specifically inhibit the synthesis of mtGPAT1 protein (typically by degrading or inhibiting the mRNA and thereby prevent protein formation) in cells and experimental animals thereby facilitating functional analysis of the target or an appraisal of its usefulness as a target for therapeutic intervention.

In diagnostics the oligomers may be used to detect and quantitate mtGPAT1 expression in cell and tissues by northern blotting, in-situ hybridisation or similar techniques.

For therapeutics, an animal or a human, suspected of having a disease or disorder, which can be treated by modulating the expression of mtGPAT1 is treated by administering oligomeric compounds in accordance with this invention. Further provided are methods of treating a mammal, such as treating a human, suspected of having or being prone to a disease or condition, associated with expression of mtGPAT1 by administering a therapeutically or prophylactically effective amount of one or more of the oligomers or compositions of the invention.

The invention also provides for the use of the compound or conjugate of the invention as described for the manufacture of a medicament for the treatment of a disorder as referred to herein, or for a method of the treatment of a disorder as referred to herein.

The invention also provides for a method for treating a disorder as referred to herein said method comprising administering a compound according to the invention as herein described, and/or a conjugate according to the invention, and/or a pharmaceutical composition according to the invention to a patient in need thereof.

### Medical Indications

The oligomers and other compositions according to the invention can be used for the treatment of conditions associated with over expression or expression of mutated version of the mtGPAT1.

The invention further provides use of a compound of the invention in the manufacture of a medicament for the treatment of a disease, disorder or condition as referred to herein.

Generally stated, one aspect of the invention is directed to a method of treating a mammal suffering from or susceptible to conditions associated with abnormal levels of mtGPAT1, comprising administering to the mammal and therapeutically effective amount of an oligomer targeted to mtGPAT1 that comprises one or more LNA units.

The disease or disorder, as referred to herein, may, In some embodiments be associated with a mutation in the mtGPAT1 gene or a gene whose protein product is associated with or interacts with mtGPAT1. Therefore, in some embodiments, the target mRNA is a mutated form of the mtGPAT1 sequence.

An interesting aspect of the invention is directed to the use of an oligomer (compound) as defined herein or a conjugate as defined herein for the preparation of a medicament for the treatment of a disease, disorder or condition as referred to herein.

The methods of the invention are preferably employed for treatment or prophylaxis against diseases caused by abnormal levels of mtGPAT1.

Alternatively stated, In some embodiments, the invention is furthermore directed to a method for treating abnormal levels of mtGPAT1, said method comprising administering a oligomer of the invention, or a conjugate of the invention or a pharmaceutical composition of the invention to a patient in need thereof.

The invention also relates to an oligomer, a composition or a conjugate as defined herein for use as a medicament.

The invention further relates to use of a compound, composition, or a conjugate as defined herein for the manufacture of a medicament for the treatment of abnormal levels of mtGPAT1 or expression of mutant forms of mtGPAT1 (such as allelic variants, such as those associated with one of the diseases referred to herein).

Moreover, the invention relates to a method of treating a subject suffering from a disease or condition such as those referred to herein.

A patient who is in need of treatment is a patient suffering from or likely to suffer from the disease or disorder.

In some embodiments, the term ‘treatment’ as used herein refers to both treatment of an existing disease (e.g. a disease or disorder as herein referred to), or prevention of a disease, i.e. prophylaxis. It will therefore be recognised that treatment as referred to herein may, In some embodiments, be prophylactic.

### Embodiments

The following embodiments of the present invention may be used in combination with the other embodiments described herein.
1. An oligomer of between 10-30 nucleotides in length which comprises a contiguous nucleotide sequence of a total of between 10-30 nucleotides, wherein said contiguous nucleotide sequence is at least 80% homologous to a region corresponding to a mammalian mtGPAT1 gene or the reverse complement of an mRNA, such as SEQ ID NO: 263 or naturally occurring variant thereof.
2. The oligomer according to embodiment 1, wherein the contiguous nucleotide sequence is at least 80% homologous to a region corresponding to any of SEQ ID NO: 264, 265, 266, 267, 268, 269, 270, 271, 272, 273, 274, 275, 276, 277, 278, 279, 280, 281, 282, 283, 284, 285, 286, 287, 288, 289, and 290.
3. The oligomer according to embodiment 1 or 2, wherein the contiguous nucleotide sequence comprises no mismatches or no more than one or two mismatches with the reverse complement of the corresponding region of SEQ ID NO: 263.
4. The oligomer according to any one of embodiments 1-3, wherein the nucleotide sequence of the oligomer consists of the contiguous nucleotide sequence.
5. The oligomer according to any one of embodiments 1-4, wherein the contiguous nucleotide sequence is between 10-18 nucleotides in length.
6. The oligomer according to any one of embodiments 1-5, wherein the contiguous nucleotide sequence comprises nucleotide analogues.
7. The oligomer according to any one of embodiments 1-6, wherein the contiguous nucleotide comprises or consists of any one of SEQ ID NO's: 1-262.
8. The oligomer according to embodiment 6 or 7, wherein the nucleotide analogues are sugar modified nucleotides, such as sugar modified nucleotides selected from the group consisting of: Locked Nucleic Acid (LNA) units; 2′-O-alkyl-RNA units, 2′-OMe-RNA units, 2′-amino-DNA units, and 2′-fluoro-DNA units.
9. The oligomer according to embodiment 6 or 7, wherein the nucleotide analogues are LNA.
10. The oligomer according to any one of embodiments 6-9 which is a gapmer.
11. The oligomer according to any one of embodiments 1-10, wherein the oligomer is any one of SEQ ID NO: 2, 33, 125, 142, 147, 169, 176, 182, 214, 249, 250 and 254.
12. The oligomer according to any one of embodiments 1-11, which inhibits the expression of mtGPAT1 gene or mRNA in a cell which is expressing mtGPAT1 gene or mRNA.
13. A conjugate comprising the oligomer according to any one of embodiments 1-12, and at least one non-nucleotide or non-polynucleotide moiety covalently attached to said oligomer.
14. A pharmaceutical composition comprising the oligomer according to any one of embodiments 1-12, or the conjugate according to embodiment 13, and a pharmaceutically acceptable diluent, carrier, salt or adjuvant.
15. The oligomer according to any one of embodiments 1-12, or the conjugate according to embodiment 13, for use as a medicament, such as for the treatment of overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).
16. The use of an oligomer according to any one of the embodiments 1-12, or a conjugate as defined in embodiment 13, for the manufacture of a medicament for the treatment of overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).
17. A method of treating overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM), said method comprising administering an oligomer according to any one of the embodiments 1-12, or a conjugate according to embodiment 13, or a pharmaceutical composition according to claim 14, to a patient suffering from, or likely to suffer from overweight, obesity, fatty liver, hepatosteatosis, non alcoholic fatty liver disease (NAFLD), non alcoholic steatohepatitis (NASH), insulin resistance, and non insulin dependent diabetes mellitus (NIDDM).
18. A method for the inhibition of mtGPAT1 in a cell which is expressing mtGPAT1, said method comprising administering an oligomer according to any one of the embodiments 1-12, or a conjugate according to embodiment 13 to said cell so as to inhibit mtGPAT1 in said cell.

### EXAMPLES

LNA monomer and oligonucleotide synthesis were performed using the methodology referred to in Examples 1 and 2 of PCT/EP2007/060703.

The stability of LNA oligonucleotides in human or rat plasma is performed using the methodology referred to in Example 4 of PCT/EP2007/060703

In vitro model; RNA extraction and cDNA synthesis is performed using the methodology referred to in Example 7 of PCT/EP2007/060703

The above mentioned examples of PCT/EP2007/060703 are hereby specifically incorporated by reference.

### Example 1

### In Vitro Model: Cell Culture

The effect of antisense compounds on target nucleic acid expression can be tested in any of a variety of cell types provided that the target nucleic acid is present at measurable levels. Target can be expressed endogenously or by transient or stable transfection of a nucleic acid encoding said nucleic acid.

The expression level of target nucleic acid can be routinely determined using, for example, Northern blot analysis, Quantitative PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

LTK-D2: Mouse fibroblast cell line LTK-D2 was purchased from ATCC and cultured in DMEM (Sigma) with 10% FBS+Glutamax I+non-essential amino acids+gentamicin.

HuH7: Human liver cell line HuH7 was purchased from ATCC and cultured in Eagle MEM (Sigma) with 10% FBS+Glutamax I+non-essential amino acids+gentamicin.

### Example 2

### In Vitro Model: Treatment with Antisense Oligonucleotide

Cell culturing and transfections: 2.5×10⁵ or 4×10⁵ cells of HuH7 or LTK-D2, respectively, were seeded in each well of 6-well plates at 37° C. (5% CO₂) in growth media supplemented with 10% FBS, Glutamax I and Gentamicin. When the cells were 60-70% confluent, they were transfected in duplicates with different concentrations of oligonucleotides (0.04-25 nM) using Lipofectamine 2000 (5 μg/ml). Transfections were carried out essentially as described by Dean et al. (1994, JBC 269:16416-16424). In short, cells were incubated for 10 min. with Lipofectamine in OptiMEM followed by addition of oligonucleotide to a total volume of 0.5 ml transfection mix per well. After 4 hours, the transfection mix was removed, cells were washed and grown at 37° C. for approximately 20 hours (mRNA analysis and protein analysis in the appropriate growth medium. Cells were then harvested for protein and RNA analysis.

### Example 3

### In Vitro and In Vivo Model: Analysis of Oligonucleotide Inhibition of mtGPAT1 Expression by Real-Time PCR

Real-Time Quantitative PCR Analysis of mtGPAT1 mRNA Levels

To determine the relative mouse mtGPAT1 mRNA level in treated and untreated samples, the generated cDNA was used in quantitative PCR analysis using a 7500Fast PCR system (Applied Biosystems)

MtGPAT1 mRNA quantification of was carried out using commercially available TaqMan assays and reagents (Applied Biosystems). In brief, 4 μl of first strand cDNA (diluted 15 times in nuclease-free water) was added to 6 μl Taqman Fast Universal PCR master mix (2×) (Applied Biosystems) supplemented with 0.5 μl 20× primer probe mix (mtGPAT1 or GAPDH).

A two-fold cDNA dilution series of mock transfected cells cDNA reaction (using 2.5 times more total RNA than in samples) served as standard to ensure a linear range (Ct versus relative copy number) of the amplification. Each sample was analysed in duplicates using PCR program: 95° C. for 20 seconds followed by 40 cycles of 95° C., 3 seconds, 60° C., 30 seconds.

Relative quantities of mtGPAT1 mRNA were determined from the calculated Threshold cycle using the Sequence Detection Software (Applied Biosystems).

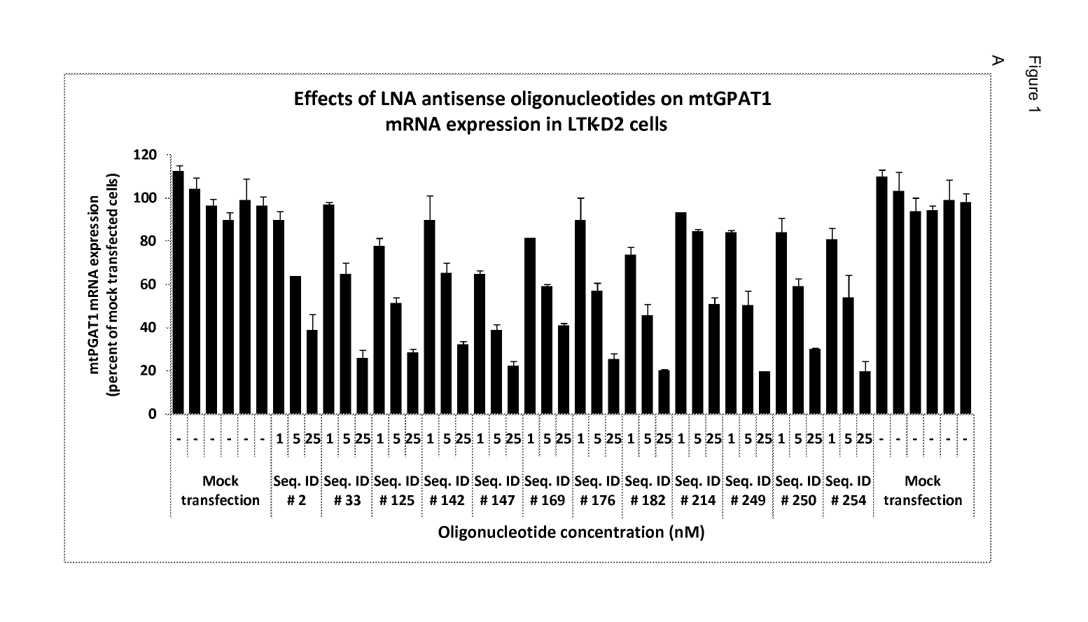

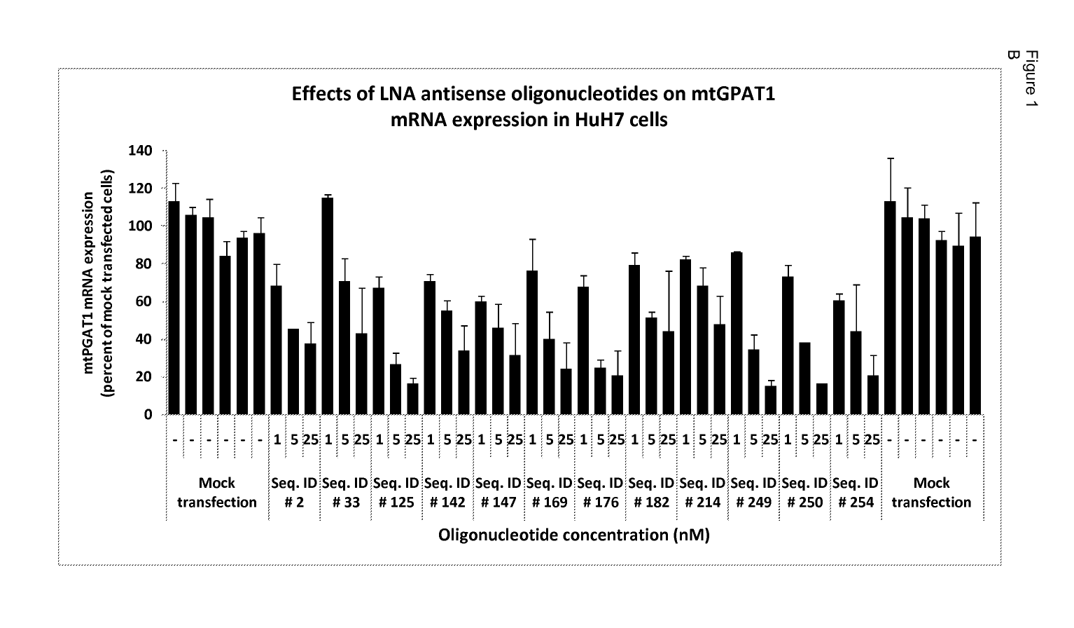

Results of analyses are illustrated in FIG. 1. The data are presented as percentage downregulation relative to mock transfected cells. Transcript steady state was monitored by Real-time PCR and normalised to the GAPDH transcript steady state.

### Example 4

### In Vivo Model; Analysis of Liver Lipid Content in Experimental Animals after Treatment with Antisense Oligonucleotides Directed Against mtGPAT1

One or several antisense oligonucleotide molecules will be selected for evaluation in in vivo experiments. The selection process includes, but is not limited to, an initial screening of efficiency of a selection of oligonucleotide molecules in terms of down-regulation of mtGPAT1 mRNA after one dose of the respective molecule (typical oligonucleotide concentration during screening is 5-25 mg/kg), followed by dose-response studies of one or several selected oligonucleotide molecules where concentration and number of doses/week are optimized to determine the lowest concentration and number of doses possible for efficient and stable down-regulation of mtGPAT1 mRNA and thereto related biological effects (see below).

Animal experiments will be performed in, but not limited to, intravascular or subcutaneous injection of antisense oligonucleotides in different mouse strains, such as C57BI/6J, NMRI, or other lipid-sensitive mice. Animals will be kept on standard chow or high fat diet for the duration of study, or on a high fat diet before starting treatment, then standard chow during the duration of treatment. A group of animals will be treated with saline, to be used as a reference/control.

After termination of experiments target organs, such as liver, will be dissected and flash-frozen in liquid nitrogen. Aliquots of respective tissue will be analyzed for mtGPAT1 mRNA and protein expression, as well as for expression of other relevant proteins. Lipid accumulation will be evaluated by HPTLC (high performance thin layer chromatography) analysis of lipid extracts of tissues. Lipid extraction will be performed using a well established standard protocol (Blight Dyer lipid extraction). Lipid accumulation will be evaluated by quantification of neutral lipids (triacylglycerol, cholesterol ester and free cholesterol) in tissue lipid extracts, with lipid content normalized to tissue mass or tissue protein content. Liver accumulation of neutral lipids at levels above control will be considered fatty livers/hepatosteatosis. Liver lipid accumulation will also be confirmed by Oil Red O staining of tissue sections, a well established technique for evaluation of tissue lipid content.

### Example 5

### In Vivo Model; Analysis of Plasma Lipid, Lipoprotein, and Inflammatory Marker Content in Experimental Animals after Treatment with Antisense Oligonucleotides Directed Against mtGPAT1

These analyses will performed in samples collected from the same experimental animals as outlined in Example 4.

During treatment, or after termination of experiments, plasma or serum from experimental animals will be collected and either analyzed directly or mixed with a cocktail of protease inhibitors and stored at −80° C. until analysis. Aliquots will be analyzed for total cholesterol and triglyceride content using colourimetric enzyme-based analyses using standard protocol according to the manufacturer's instructions (ABX Pentra, Horiba, France). Samples will also be analyzed for lipoprotein lipid distribution, again using standard protocol according to the manufacturer's instructions (Sebia, France).

Lipid accumulation in tissues may start inflammatory reactions, a process often referred to as part of lipotoxicity. Quantification of secretion of pro-inflammatory cytokines to serum/plasma can be used as a means of monitoring of tissue inflammation. Levels of pro- and anti-inflammatory cytokines in serum or plasma from experimental animals will be analyzed by ELISA or by Luminix (Luminix,) methods using standard protocols according to the manufacturer's instructions. Cytokine analysis will include quantification of plasma or serum levels of TNF-α, IL-1β, IL-6 and SAA.

### Example 6

### In Vivo Downregulation of Liver mtGPAT mRNA Expression in Female C57BL/6 Mice

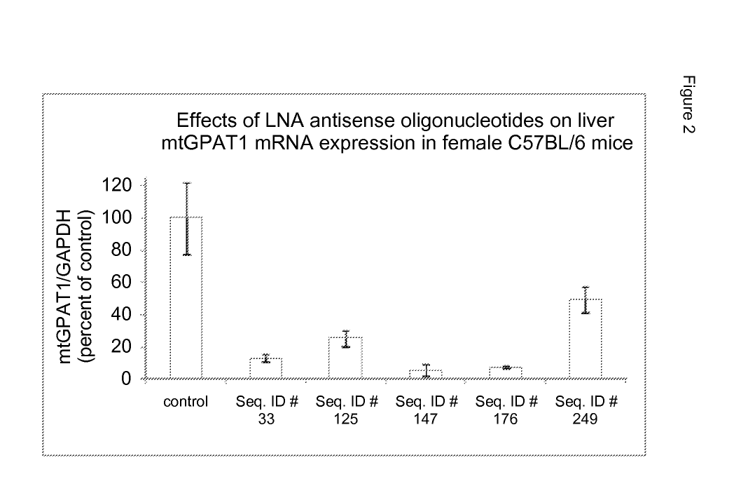

The effect of 5 different mtGPAT antisense oligomers, SEQ ID # 33, 125, 147, 176, and 249 on liver mtGPAT mRNA expression was tested. Female C57BL/6 mice were injected three times (days 0, 3, and 7) with respective compound at 15 mg/kg before termination of experiment at day 9, 48 h after the last injection. Liver mRNA was isolated and RT-PCR for mtGPAT1 and GAPDH was performed after cDNA synthesis. Data as shown in FIG. 2 are expressed as mtGPAT1/GAPDH mRNA concentration as percent of mtGPAT1/GAPDH mRNA concentration in control animals injected with saline.

SEQUENCE LISTING

<212> 263
<211> 6390
<212> DNA
<213> homo sapiens
<400> 2

| | SEQ ID NO: 263 |
|---|---|
| 1 | gtgcgccact gcagctggca ttggccggga ctggaagtgc gggcttctgc agcagccgaa |
| 61 | gctggagctg ctaggcagcg gctcccctgt tgtatggaca ttctgcaccc gaaactgata |
| 121 | gctgagtcct gaagttttat gttatgaaac agaagaactt tcatcccagc acatgatttg |
| 181 | ggaattacac tttgtgacat ggatgaatct gcactgaccc ttggtacaat agatgtttct |
| 241 | tatctgccac attcatcaga atacagtgtt ggtcgatgta agcacacaag tgaggaatgg |
| 301 | ggtgagtgtg gctttagacc caccatcttc agatctgcaa ctttaaaatg gaaagaaagc |
| 361 | ctaatgagtc ggaaaaggcc atttgttgga agatgttgtt actcctgcac tccccagagc |
| 421 | tgggacaaat ttttcaaccc cagtatcccg tctttgggtt tgcggaatgt tatttatatc |
| 481 | aatgaaactc acacaagaca ccgcggatgg cttgcaagac gcctttctta cgttcttttt |
| 541 | attcaagagc gagatgtgca taagggcatg tttgccacca atgtgactga aaatgtgctg |
| 601 | aacagcagta gagtacaaga ggcaattgca gaagtggctg ctgaattaaa ccctgatggt |
| 661 | tctgcccagc agcaatcaaa agccgttaac aaagtgaaaa agaaagctaa aaggattctt |
| 721 | caagaaatgg ttgccactgt ctcaccggca atgatcagac tgactgggtg ggtgctgcta |
| 781 | aaactgttca acagcttctt ttggaacatt caaattcaca aaggtcaact tgagatggtt |
| 841 | aaagctgcaa ctgagacgaa tttgccgctt ctgtttctac cagttcatag atcccatatt |
| 901 | gactatctgc tgctcacttt cattctcttc tgccataaca tcaaagcacc atacattgct |
| 961 | tcaggcaata atctcaacat cccaatcttc agtaccttga tccataagct tgggggcttc |
| 1021 | ttcatacgac gaaggctcga tgaaacacca gatggacgga aagatgttct ctatagagct |
| 1081 | ttgctccatg ggcatatagt tgaattactt cgacagcagc aattcttgga gatcttcctg |
| 1141 | gaaggcacac gttctaggag tggaaaaacc tcttgtgctc gggcaggact tttgtcagtt |
| 1201 | gtggtagata ctctgtctac caatgtcatc ccagacatct tgataatacc tgttggaatc |
| 1261 | tcctatgatc gcattatcga aggtcactac aatggtgaac aactgggcaa acctaagaag |
| 1321 | aatgagagcc tgtggagtgt agcaagaggt gttattagaa tgttacgaaa aaactatggt |
| 1381 | tgtgtccgag tggattttgc acagccattt tccttaaagg aatatttaga aagccaaagt |
| 1441 | cagaaaccgg tgtctgctct actttccctg gagcaagcgt tgttaccagc tatacttcct |
| 1501 | tcaagaccca gtgatgctgc tgatgaaggt agagacacgt ccattaatga gtccagaaat |
| 1561 | gcaacagatg aatccctacg aaggaggttg attgcaaatc tggctgagca tattctattc |
| 1621 | actgctagca agtcctgtgc cattatgtcc acacacattg tggcttgcct gctcctctac |
| 1681 | agacacaggc agggaattga tctctccaca ttggtcgaag acttctttgt gatgaaagag |
| 1741 | gaagtcctgg ctcgtgattt tgacctgggg ttctcaggaa attcagaaga tgtagtaatg |
| 1801 | catgccatac agctgctggg aaattgtgtc acaatcaccc acactagcag gaacgatgag |
| 1861 | ttttttatca cccccagcac aactgtccca tcagtcttcg aactcaactt ctacagcaat |
| 1921 | ggggtacttc atgtctttat catggaggcc atcatagctt gcagccttta tgcagttctg |
| 1981 | aacaagaggg gactgggggg tcccactagc accccaccta acctgatcag ccaggagcag |
| 2041 | ctggtgcgga aggcggccag cctgtgctac cttctctcca atgaaggcac catctcactg |
| 2101 | ccttgccaga cattttacca agtctgccat gaaacagtag gaaagtttat ccagtatggc |
| 2161 | attcttacag tggcagagca cgatgaccag gaagatatca gtcctagtct tgctgagcag |
| 2221 | cagtgggaca agaagcttcc agaacctttg tcttggagaa gtgatgaaga agatgaagac |
| 2281 | agtgactttg gggaggaaca gcgagattgc tacctgaagg tgagccaatc caaggagcac |
| 2341 | cagcagttta tcaccttctt acagagactc cttgggcctt tgctggaggc ctacagctct |
| 2401 | gctgccatct ttgttcacaa cttcagtggt cctgttccag aacctgagta tctgcaaaag |
| 2461 | ttgcacaaat acctaataac cagaacagaa agaaatgttg cagtatatgc tgagagtgcc |
| 2521 | acatattgtc ttgtgaagaa tgctgtgaaa atgtttaagg atattggggt tttcaaggag |
| 2581 | accaaacaaa agagagtgtc tgttttagaa ctgagcagca cttttctacc tcaatgcaac |
| 2641 | cgacaaaaac ttctagaata tattctgagt tttgtggtgc tgtaggtaac gtgtggcact |
| 2701 | gctggcaaat gaaggtcatg agatgagttc cttgtaggta ccagcttctg gctcaagagt |
| 2761 | tgaaggtgcc atcgcagggt caggcctgcc ctgtcccgaa gtgatctcct ggaagacaag |
| 2821 | tgccttctcc ctccatggat ctgtgatctt cccagctctg catcaacaca gcagcctgca |
| 2881 | gataacactt ggggggacct cagcctctat tcgcaactca taatccgtag actacaagat |
| 2941 | gaaatctcaa taaattattt ttgagtttat taaagattga cattttaagt acaactttta |
| 3001 | aggactaatt actgtgatgg acacagaaat gtagctgtgt tctggaactg aatcttacat |
| 3061 | ggtatactta gtgctgctgg gtaatttgtt ggtatattat ctggttagtg gttaatgctt |
| 3121 | cctttaaaaa taattgagtc atccattcac tctttttcag ttttatctgt caatagtagc |
| 3181 | tacattttta atgggagcac cttttatccc aaagtgcttt ataaattgag tggactgata |
| 3241 | tatatcacac ccaggtatca ctgtgctgtc ctttgctgtc agatttagaa atgtttttaa |
| 3301 | gagctatgtg aaaacagaca atattagttt aggtcgggaa ctgagatatt gtaatcaaat |
| 3361 | agttaacatc aggaagttaa tttggctggc aaaattctag ggaaacttgg ccagaaaact |
| 3421 | ggtgttgaag gcttttgctc atataaacaa gtgccattga gtttcaaatg accagcaaat |
| 3481 | atatttagaa cccttcctgt tttatgtctg tacctcgtcc acccctcagg taatacctgc |
| 3541 | ctctcacagg tacagctgtt tcttggaaat cctccaacca aatagcagtt ttcctaactt |
| 3601 | gattagcttg agctgacaga ctgttagaat acagttctct ggccacagct gatgagggct |
| 3661 | ttctgtactg cacacagatt gtgtactgca ccccagtcca ggtgactggt acccactcga |
| 3721 | gttgtgccgt gcacaacctg tccagtatat gcatgtggtg gccctactga ctggtaatgg |
| 3781 | ttagaggcat ttatggattt ttagctttga ggaaaaacca tgacttttaa caaattttta |
| 3841 | tgggttatat gcctaaaccc ttatgccaca tagtggtaaa taattatgaa aaatggtctg |
| 3901 | ttcataattg gtaggtgcct tttgtgagca gggagcataa ttattggttt attatggtaa |
| 3961 | ttatggtgat tttttaaata tcatgtaatg ttaaaacgtt ttctaacagt ttactgttgc |
| 4021 | ttatctccaa gatattatgg aattaagaat ttttccagat gagtgttaca tagattcttt |
| 4081 | gaatttagta taaaagtact gagaattaag tttgtacttc cataagcttg gattttaaac |
| 4141 | actgatagta tctcatgagt aatgtgtgtt ttgggagagg gagggatgct gattgatatt |
| 4201 | tcacattgta tgaaatacca tgtttgaaac tcatagcaat aatgctatgc tgttgtgatc |
| 4261 | cctctcaagt tctgcattta aaatatattt tttctttata ggaattgatg tataccatga |
| 4321 | agtcattgtc agttgtagta gctctgatgt tgaatgagat atcatgtttt agcattccat |
| 4381 | tttactgact agggtagaag aacacttttc ttggctacat ttggaggata cccagggagt |
| 4441 | cttgggtgtt ccttatctgg ggaagcaaac atttcactag tctctttttt tcatccttta |
| 4501 | aattgtaaat taaggattac tcaagctcac cattattcaa gattgggact cgcttcccag |
| 4561 | tcgacactct gccctgcctg tcattgctgc aaagagctgc tgctttgcca acctaagcaa |
| 4621 | agaaaatacg gcttctcttg cattattttc ccttttggtt ggtttgtttt ctagaagtac |
| 4681 | gttcagatgc tttggggaat gcaatgtatg atttgctagc tctctcacca cttaactcac |
| 4741 | tgtgaggata aatatgcatg ctttttgtaa ttaactggtg ctttgaaaat cttttttaag |
| 4801 | ggagaaaaat ctcaaccaaa gttatgctca tccagacaag ctgacctttg agttaatttc |
| 4861 | agcacaactc attcttcagt gcctcatgac tgaaaacaaa aaacaaaaaa acgaaagcat |
| 4921 | cttcacaatg aagcttccag atagcaccgt tttgctaaaa gatacattct cattgttttc |
| 4981 | caacagtgat ggcttccaca taaggttaaa caaactaggt gcttgtaaat aatttattac |
| 5041 | agtttactct atcgcatttc tgtaacatga aatgcatgcc cttcttcagg ggaagactgt |
| 5101 | ggtcaagtta aaaaaaaaaa acaatattaa acaacatgaa actgcagtct gtttttgaaa |
| 5161 | atgagaatgt cctaagtgat tcagaagaga ggagggaagt tgtgcactct gaaaatgcat |
| 5221 | gaaaaacaaa ggcaaaaact agtgggaaat gtgtagaact gttaactgag atggcttcga |
| 5281 | gtcttccttc tggaatctgt taaatttcac aaagtcatga gggtaaatgg agaaaatatt |
| 5341 | tctgggatta caatgaatgt aagcccaaat tgtggaattg ccagtaacct ggatggggaa |
| 5401 | aagcatttcc catagcactc catgtaatat gagtgctctg tgagatgttc atcagtgttt |
| 5461 | tatagaaatg gtgttgctgg gaaaccaagt ttgcacctgg aaacttacaa tgcactttag |
| 5521 | cgcagtaagg gcttggcatc cggtagtgaa aaactgtcta acccagcatt gcccaaacta |
| 5581 | ttttgacacc aggacctttt tctcctttgg gatacttatg aacctctcac taatgtcctg |
| 5641 | tggagaacat tttgggaaac actatgttag atagttcttt aaggagacaa aacggtaatg |
| 5701 | aacagatagc actggggcag aatatgcatg cattttgtaa cgtccagtgt ggcgttgaat |
| 5761 | agatgtgtat ttcctcccct gcagaaaata agcacagaaa attataatgt aggtgatcgg |
| 5821 | agctctttcc tttgatagag agaacagccc caatgatcct ggctttttca ctgaacgtat |
| 5881 | cagaatacat ggatgaattg gggtaaataa ggttttaatt cagatctaga agaaagtatt |
| 5941 | gtacgtttga atgcagattt ttatccacag atagttgtag tgtttagaca tgacaggacc |
| 6001 | tatcgttgag gtttctaaga cttactatgg gctgtaaacc tgttttttaa aactatttta |
| 6061 | gaaacctgag acttgccgtc tggcatttta gtttaataca aactaatgat tgcatttgaa |
| 6121 | agagattctt gaccttattt ctaaacgtct agagctctga aatgtcttga tggaaggtat |
| 6181 | taaactattt gcctgttgta caaagaaatg ttaagactcg tgaaaagaat tactataagg |
| 6241 | tactgtgaaa taactgcgat tttgtgagca aaacatactt ggaaatgctg attgattttt |
| 6301 | atgcttgtta gtgtattgca agaaacacag aaaatgtagt tttgttttaa taaaccaaaa |
| 6361 | attgaacata caaaaaaaaa aaaaaaaaaa |

1-18. (canceled)

19. An oligomer 10 to 30 nucleotides in length which comprises a contiguous nucleotide sequence of 10 to 30 nucleotides, wherein the contiguous nucleotide sequence is at least 80% homologous to a region corresponding to a mammalian mtGPAT1 gene or the reverse complement of an mRNA encoding a mammalian mtGPAT1, or naturally occurring variant thereof.

20. The oligomer according to claim 19 wherein the contiguous nucleotide sequence is at least 80% homologous to SEQ ID NO:263.

21. The oligomer according to claim 19, wherein the contiguous nucleotide sequence is at least 80% homologous to a region corresponding to any of SEQ ID NO: 264, 265, 266, 267, 268, 269, 270, 271, 272, 273, 274, 275, 276, 277, 278, 279, 280, 281, 282, 283, 284, 285, 286, 287, 288, 289, and 290.

22. The oligomer according to claim 19, wherein the contiguous nucleotide sequence comprises no mismatches or no more than one or two mismatches with the reverse complement of the corresponding region of SEQ ID NO: 263.

23. The oligomer according to claim 19, wherein the nucleotide sequence of the oligomer consists of the contiguous nucleotide sequence.

24. The oligomer according to claim 19, wherein the contiguous nucleotide sequence is 10 to 18 nucleotides in length.

25. The oligomer according to claim 19, wherein the contiguous nucleotide sequence comprises nucleotide analogues.

26. The oligomer according to claim 19, wherein the contiguous nucleotide comprises or consists of any one of SEQ ID NO's: 1-262 and 291-302.

27. The oligomer according to claim 25, wherein the nucleotide analogues are sugar modified nucleotides selected from the group consisting of: Locked Nucleic Acid (LNA) units; 2′-O-alkyl-RNA units, 2′-OMe-RNA units, 2′-amino-DNA units, and 2′-fluoro-DNA units.

28. The oligomer according to claim 27, wherein the nucleotide analogues are LNA.

29. The oligomer according to claim 25 which is a gapmer.

30. The oligomer according to claim 19, wherein the oligomer is any one of SEQ ID NO: 2 & 291, 33 & 292, 125 & 293, 142 & 294, 147 & 295, 169 & 296, 176 & 297, 182 & 298, 214 & 299, 249 & 300, 250 & 301 and 254 & 302.

31. The oligomer according to claim 19, which inhibits the expression of mtGPAT1 gene or mRNA in a cell which is expressing mtGPAT1 gene or mRNA.

32. A conjugate comprising the oligomer according to claim 19, and at least one non-nucleotide or non-polynucleotide moiety covalently attached to the oligomer.

33. A pharmaceutical composition comprising the oligomer according to claim 19 or a conjugate according to claim 32, and a pharmaceutically acceptable diluent, carrier, salt or adjuvant.

34. A method of treating a subject suffering from a condition selected from the group consisting of: excess bodyweight, obesity, fatty liver, hepatosteatosis, non-alcoholic fatty liver disease (NAFLD), non-alcoholic steatohepatitis (NASH), insulin resistance, and non-insulin dependent diabetes mellitus (NIDDM), the method comprising administering an effective amount of an oligomer according to claim 19 or an conjugate according to claim 32 to the subject.
